(19) United States
(12) Patent Application Publication
(10) Pub. No.: US 2012/0128743 A1
(43) Pub. Date: May 24, 2012

(54) POLYMER PARTICLES

(76) Inventors: Brian Stanley Hawkett, Mona Vale (AU); Thi Thuy Binh Pham, Rydalmere (AU); Christopher Henry Such, Mount Eliza (AU)

(21) Appl. No.: 13/202,880

(22) PCT Filed: Feb. 24, 2010
(86) PCT No.: PCT/AU2010/000212

§ 371 Date: Feb. 6, 2012

(30) Foreign Application Priority Data
Feb. 24, 2009 (AU) 2009900799
Jul. 3, 2009 (AU) 2009903131

(51) Int. Cl. A61K 8/02; C08L 33/12; C09D 133/12; C09D 125/06; C09J 133/12; C08L 33/02; C08L 33/26; A61K 9/14; C08L 25/06; C09J 125/06; B82B 3/00; B82Y 30/00

(52) U.S. Cl. 424/401; 525/241; 525/227; 524/523; 524/515; 525/299; 525/294; 424/497; 977/773; 977/892

(57) ABSTRACT

The present invention relates to a method of forming polymer on the surface of polymer particles, the method comprising:
(i) providing a dispersion comprising a continuous aqueous phase, a dispersed organic phase comprising one or more ethylenically unsaturated monomers, and a RAFT agent as a stabiliser for said organic phase;
(ii) polymerising the one or more ethylenically unsaturated monomers under the control of the RAFT agent to form an aqueous dispersion of seed polymer particles;
(iii) crosslinking the seed polymer particles;
(iv) swelling the crosslinked seed particles with one or more ethylenically unsaturated monomers to form an aqueous dispersion of monomer swollen crosslinked seed polymer particles;
(v) increasing the temperature of the monomer swollen crosslinked seed polymer particles to expel at least some of the monomer therein onto the surface of the particles; and
   polymerising at least the expelled monomer to form polymer on the surface of the particles.

### FIELD OF THE INVENTION

The present invention relates in general to polymer particles, and in particular to a method of forming polymer on the surface of polymer particles. The invention also relates to unique polymer particles, to products comprising polymer particles, and to using polymer particles in the manufacture of a dispersion of polymer particles.

### BACKGROUND OF THE INVENTION

Polymer particles are used extensively in a diverse array of applications. For example, they may be used in coatings (e.g. paint), adhesive, filler, primer, sealant, pharmaceutical, cosmetic and diagnostic applications.

In recent years there has been increased interest in the development and use of micron or sub-micron heterogeneous polymer particles (i.e. polymer particles comprising at least two sections or regions of polymer that each have a different molecular composition). Heterogeneous polymer particles include those having core-shell and non-core-shell structures.

Heterogeneous core-shell polymer structures are known in the art to comprise a substantially spherical core polymer region that is encapsulated by a shell polymer region, with the core and shell polymer regions having different molecular compositions. Such structures typically present only one exposed polymer composition, namely the shell polymer composition, with the core polymer composition being internalised by the encapsulating shell polymer.

As used herein, an “exposed” polymer composition is intended to mean a polymer composition that is adjacent to or in contact with an environment external to the polymer particles. For example, where the polymer particles are dispersed in a liquid, an exposed polymer composition will be one that is directly adjacent or in contact with the liquid.

Heterogeneous core-shell polymer particles that present an outer (i.e. shell) polymer composition that is different from the inner (i.e. core) polymer composition can exhibit properties associated with each polymer component, but the internalised core composition can advantageously be masked from an external environment by the shell composition. For example, the particles may have a relatively hard abrasion resistant polymer core and a relatively soft film forming polymer shell, the likes which may be used to prepare unique coatings compositions.

Numerous techniques have been developed for preparing core-shell polymer structures. However, in practice it has been difficult to date to produce particles having a substantially uniform and/or continuous polymer shell encapsulating a polymer core.

Heterogeneous non-core-shell polymer structures are known in the art to comprise at least two polymer regions or sections of different molecular compositions that are associated but not in a core-shell structure. Heterogeneous non-core-shell polymer structures therefore necessarily present at least two exposed polymer regions or sections of different molecular composition and can take a variety of physical forms.

Due to the presence of at least two exposed polymer regions or sections of different molecular composition, heterogeneous non-core-shell polymer structures are often referred to as anisotropic polymer particles. The anisotropic nature of such particles can give rise to asymmetric interactions that can advantageously impart unique properties.

Numerous techniques have also been developed for preparing heterogeneous non-core-shell polymer structures. However, in practice it has been difficult to date to control the morphology, size and composition of such particles.

A particular class of heterogeneous non-core-shell polymer structures of emerging interest include those which present two surfaces or faces of different composition or structure (known in the art as Janus particles). Janus character is therefore a surface rather than bulk property of the particles. Accordingly, anisotropic polymer particles may not necessarily exhibit Janus character. In that case, despite anisotropic polymer particles having at least two exposed polymer regions or sections of different molecular composition, the surface of each region or section in contact with the external environment can be relatively indistinguishable. For example, the particles may be stabilised in a liquid with a surface active agent that modifies the entire surface character of the particles. In such an example, the at least two exposed polymer regions or sections of different molecular composition are “adjacent” the liquid and the surface active agent is “in contact” with the liquid.

Conventional techniques for preparing micron or sub-micron Janus particles often suffer from extremely low yields, thereby limiting their practical application. Techniques have been developed for producing larger quantities of Janus particles, but these generally afford relatively large particles (e.g. several microns in diameter).

An opportunity therefore remains to address or ameliorate one or more disadvantages or shortcomings associated with existing polymer particles and techniques for preparing such particles, or to at least provide useful alternative polymer particles and technique for their preparation.

### SUMMARY OF THE INVENTION

The present invention therefore provides a method of forming polymer on the surface of polymer particles, the method comprising:
(i) providing a dispersion comprising a continuous aqueous phase, a dispersed organic phase comprising one or more ethylenically unsaturated monomers, and a RAFT agent as a stabiliser for said organic phase;
(ii) polymerising the one or more ethylenically unsaturated monomers under the control of the RAFT agent to form an aqueous dispersion of seed polymer particles;
(iii) crosslinking the seed polymer particles;
(iv) swelling the crosslinked seed particles with one or more ethylenically unsaturated monomers to form an aqueous dispersion of monomer swollen crosslinked seed polymer particles;
(v) increasing the temperature of the monomer swollen crosslinked seed polymer particles to expel at least some of the monomer therein onto the surface of the particles; and
(vi) polymerising at least the expelled monomer to form polymer on the surface of the particles.

The method in accordance with the invention can advantageously be performed on a small laboratory scale or on a large industrial scale and in both cases afford high yields. The method may also be used to prepare micron and sub-micron (e.g. less than 1000 nm, such as less than 100 nm) heterogeneous core-shell and non-core-shell polymer particles with excellent control over particle composition, size and morphology.

The method in effect includes two polymerisation stages whereby in a first stage monomer is polymerised and resulting polymer chains crosslinked to form crosslinked seed polymer particles, and in a second stage monomer is polymerised on the surface of the crosslinked seed particles. The polymer formed on the surface of the crosslinked seed particles will generally have a different molecular composition to that of the seed particles. In that case, the resulting polymer particles may be described as being heterogeneous polymer particles. By controlling the manner in which the monomer swollen crosslinked seed polymer particles expel monomer, the method can advantageously be used to prepare core-shell and non-core-shell polymer particles.

Core-shell polymer particles may be formed where monomer is expelled so as to coat substantially the entire seed particle surface, whereas non-core-shell polymer particles may be formed where monomer is expelled to coat only a proportion (i.e. a fraction) of the particle surface.

In one embodiment, increasing the temperature of the monomer swollen crosslinked seed polymer particles expels at least some of the monomer therein onto substantially the entire surface of the particles, and polymerisation of at least the expelled monomer results in the formation of core-shell polymer particles.

In a further embodiment, increasing the temperature of the monomer swollen crosslinked seed polymer particles expels at least some of the monomer therein only onto a proportion of the surface of the particles, and polymerisation of at least the expelled monomer results in the formation of non-core-shell polymer particles.

The present invention therefore also provides a method of preparing core-shell polymer particles, the method comprising:
(i) providing a dispersion comprising a continuous aqueous phase, a dispersed organic phase comprising one or more ethylenically unsaturated monomers, and a RAFT agent as a stabiliser for said organic phase;
(ii) polymerising the one or more ethylenically unsaturated monomers under the control of the RAFT agent to form an aqueous dispersion of seed polymer particles;
(iii) crosslinking the seed polymer particles;
(iv) swelling the crosslinked seed particles with one or more ethylenically unsaturated monomers to form an aqueous dispersion of monomer swollen crosslinked seed polymer particles;
(v) increasing the temperature of the monomer swollen crosslinked seed polymer particles to expel at least some of the monomer therein onto substantially the entire surface of the particles; and
(vi) polymerising at least the expelled monomer to form the core-shell particles.

The present invention further provides a method of preparing non-core-shell polymer particles, the method comprising:
(i) providing a dispersion comprising a continuous aqueous phase, a dispersed organic phase comprising one or more ethylenically unsaturated monomers, and a RAFT agent as a stabiliser for said organic phase;
(ii) polymerising the one or more ethylenically unsaturated monomers under the control of the RAFT agent to form an aqueous dispersion of seed polymer particles;
(iii) crosslinking the seed polymer particles;
(iv) swelling the crosslinked seed particles with one or more ethylenically unsaturated monomers to form an aqueous dispersion of monomer swollen crosslinked seed polymer particles;
(v) increasing the temperature of the monomer swollen crosslinked seed polymer particles to expel at least some of the monomer therein onto only a proportion of the surface of the particles; and
(vi) polymerising at least the expelled monomer to form the non-core-shell particles.

In one embodiment, the non-core-shell polymer particles prepared in accordance with the invention are Janus polymer particles.

In another embodiment, crosslinking of the seed polymer particles takes place simultaneously with the seed particles being formed (i.e. steps (ii) and (iii) occur simultaneously).

In a further embodiment, crosslinking of the seed polymer particles takes place after the seed particles have been formed (i.e. steps (ii) and (iii) occur separately).

The methods in accordance with the invention may be used to prepare polymer particles having a diverse range in size. For example, the particles may have a largest dimension of no more than about one micron, for example of no more than about 500 nm, of no more than about 100 nm, of no more than about 70 nm, of no more than about 50 nm, and even of no more than about 40 nm.

The methods in accordance with the invention are particularly well suited for preparing core-shell and non-core shell polymer particles having a largest dimension of no more than about 100 nm, of no more than about 70 nm, of no more than about 50 nm, and even of no more than about 40 nm.

The methods of the invention advantageously afford an aqueous dispersion of polymer particles. The dispersion can be used in a variety of applications including the manufacture of coatings compositions.

The methods of the invention are believed to afford unique polymeric particles.

The present invention therefore also provides polymer particles capable of being dispersed in a liquid, the particles comprising two polymer regions of different molecular composition, wherein one of the polymer regions is a crosslinked RAFT polymer having covalently bound to its surface RAFT polymer chains that function as a stabiliser for the particles when they are dispersed in the liquid.

In one embodiment, the polymer particles are self stabilising polymer particles.

Further aspects of the invention are discussed in more detail below.

### BRIEF DESCRIPTION OF THE DRAWINGS

Preferred embodiments of the invention will now be illustrated by way of example only with reference to the accompanying drawings in which:

FIG. 1 is an illustration of surface wetting characteristics between expelled monomer and crosslinked seed polymer particles where it is (a) very favourable, (b) mildly favourable, (c) not very favourable, and (d) not at all favourable, for the expelled monomer to wet the crosslinked seed polymer surface;

FIG. 2 illustrates football shaped non-core-shell polymer particles prepared in accordance with the invention; and

FIG. 3 illustrates dumbbell shaped non-core-shell polymer particles prepared in accordance with the invention.

### DETAILED DESCRIPTION OF THE INVENTION

The methods in accordance with the invention can advantageously be performed using conventional dispersion polymerisation techniques (e.g. conventional emulsion, mini-emulsion and suspension polymerisation) and equipment.

The methods comprise providing a dispersion having a continuous aqueous phase, a dispersed organic phase comprising one or more ethylenically unsaturated monomers, and a RAFT agent as a stabiliser for the organic phase.

The dispersion may be simplistically described as an aqueous phase having droplets of organic phase dispersed therein. In this context, the term “phase” is used to convey that there is an interface between the aqueous and organic media formed as a result of the media being substantially immiscible.

In isolation, it will be appreciated that the aqueous and organic phases will typically be an aqueous and organic medium (e.g. liquid), respectively. In other words, the term “phase” simply assists with describing these media when provided in the form of a dispersion. However, for convenience the aqueous and organic media used to prepare the dispersion may hereinafter simply be referred to as the aqueous and organic phases, respectively.

In addition to the organic phase and the RAFT agent, the continuous aqueous phase may comprise one or more other components. For example, the aqueous phase may also comprise one or more aqueous soluble solvents and one or more additives such as those that can regulate and/or adjust pH.

In addition to the one or more ethylenically unsaturated monomers, the dispersed organic phase may comprise one or more other components. For example, the dispersed organic phase may also comprise one or more solvents that are soluble in the monomers, and/or one or more plasticisers. Solvent soluble in the monomer may act as a plasticiser.

As will be discussed in more detail below, the one or more ethylenically unsaturated monomers in the dispersed organic phase are polymerised to form seed polymer particles. The seed polymer particles are also crosslinked. Provided crosslinked seed polymer particles can be formed, there is no particular limitation on the type of ethylenically unsaturated monomers that may be used in accordance with the invention.

Suitable ethylenically unsaturated monomers are those which can be polymerised by a free radical process. The monomers should also be capable of being polymerised with other monomers. The factors which determine copolymerisability of various monomers are well documented in the art. For example, see: Greenlee, R. Z., in Polymer Handbook 3^{rd} Edition (Brandup, J., and Immergut. E. H. Eds) Wiley: New York, 1989 p II/53. Such monomers include those with the general formula (I):

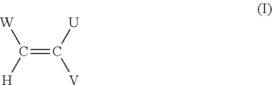

where U and W are independently selected from the group consisting of —CO₂H, —CO₂R¹, —COR¹, —CSR¹, —CSOR¹, —COSR¹, —CONH₂, —CONR¹₂, hydrogen, halogen and optionally substituted C₁-C₄ alkyl, or U and W form together a lactone, anhydride or imide ring that may itself be optionally substituted, wherein the substituents are independently selected from the group consisting of hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹; and
V is selected from the group consisting of hydrogen, R¹, —CO₂H, —CO₂R¹, —COR¹, —CSR¹, —CSOR¹, —COSR¹, —CONH₂, —CONHR¹, —CONR¹₂, —OR¹, —SR¹, —O₂CR¹, —SCOR¹, and —OCSR¹;
where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

The or each R¹ may also be independently selected from optionally substituted C₁-C₂₂ alkyl, optionally substituted C₂-C₂₂ alkenyl, optionally substituted C₂-C₂₂ alkynyl, optionally substituted C₆-C₁₈ aryl, optionally substituted C₃-C₁₈ heteroaryl, optionally substituted C₃-C₁₈ carbocyclyl, optionally substituted C₂-C₁₈ heterocyclyl, optionally substituted C₇-C₂₄ arylalkyl, optionally substituted C₄-C₁₈ heteroarylalkyl, optionally substituted C₇-C₂₄ alkylaryl, optionally substituted C₄-C₁₈ alkylheteroaryl, and an optionally substituted polymer chain.

The or each R¹ may also be selected from optionally substituted C₁-C₁₈ alkyl, optionally substituted C₂-C₁₈ alkenyl, optionally substituted aryl, optionally substituted heteroaryl, optionally substituted carbocyclyl, optionally substituted heterocyclyl, optionally substituted aralkyl, optionally substituted heteroarylalkyl, optionally substituted alkaryl, optionally substituted alkylheteroaryl and a polymer chain.

In one embodiment, the or each R¹ may be independently selected from optionally substituted C₁-C₆ alkyl.

Examples of optional substituents for R¹ include those selected from alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amino, including salts and derivatives thereof. Examples polymer chains include those selected from polyalkylene oxide, polyarylene ether and polyalkylene ether.

R¹ may also be selected from the group consisting of optionally substituted C₁-C₁₈ alkyl, optionally substituted C₂-C₁₈ alkenyl, optionally substituted aryl, optionally substituted heteroaryl, optionally substituted carbocyclyl, optionally substituted heterocyclyl, optionally substituted aralkyl, optionally substituted heteroarylalkyl, optionally substituted alkaryl, optionally substituted alkylheteroaryl and polymer chains wherein the substituents are independently selected from the group consisting of alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amino, including salts and derivatives thereof. Preferred polymer chains include, but are not limited to, polyalkylene oxide, polyarylene ether and polyalkylene ether.

Some examples of suitable ethylenically unsaturated monomers include maleic anhydride, N-alkylmaleimide, N-arylmaleimide, dialkyl fumarate and cyclopolymerisable monomers, acrylate and methacrylate esters, acrylic and methacrylic acid, styrene, acrylamide, methacrylamide, and methacrylonitrile, mixtures of these monomers, and mixtures of these monomers with other monomers.

Further examples of useful ethylenically unsaturated monomers include: methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacrylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropylacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, N-vinylcarbazole, butadiene, ethylene and chloroprene. This list is not exhaustive.

In some embodiments of the invention the ethylenically unsaturated monomers may comprise one or more ionisable ethylenically unsaturated monomers. By the term “ionisable” or “ionic” used in connection with ethylenically unsaturated monomers or a group or region of a macro RAFT agent formed using such monomers, is meant that the monomer, group or region has a functional group which can be ionised to form a cationic or anionic group. Such functional groups will generally be capable of being ionised under acidic or basic conditions through loss or acceptance of a proton. Generally, the ionisable functional groups are acid groups or basic groups. For example, a carboxylic acid functional group may form a carboxylate anion under basic conditions, and an amine functional group may form a quaternary ammonium cation under acidic conditions. The functional groups may also be capable of being ionised through an ion exchange process.

By the term “non-ionisable” or “non-ionic”, used in connection with ethylenically unsaturated monomers or a group or region of a macro RAFT agent formed using such monomers, is meant that the monomer, group or region does not have ionisable functional groups. In particular, such monomers, groups or regions do not have acid groups or basic groups which can loose or accept a proton under acidic or basic conditions.

Examples of ionisable ethylenically unsaturated monomers which have acid groups include, but are not limited to, methacrylic acid, acrylic acid, itaconic acid, p-styrene carboxylic acids, p-styrene sulfonic acids, vinyl sulfonic acid, vinyl phosphonic acid, ethacrylic acid, alpha-chloroacrylic acid, crotonic acid, fumaric acid, citraconic acid, mesaconic acid and maleic acid. Examples of ionisable ethylenically unsaturated monomers which have basic groups include, but are not limited to, 2-(dimethyl amino) ethyl and propyl acrylates and methacrylates, and the corresponding 3-(diethylamino) ethyl and propyl acrylates and methacrylates. Examples of non-ionisable hydrophilic ethylenically unsaturated monomers include, but are not limited to, hydroxy ethyl methacrylate, hydroxy propyl methacrylate, and hydroxy ethyl acrylate.

Further detail regarding ethylenically unsaturated monomers that may be used in accordance with the invention is outlined below in a discussion on crosslinking of the seed polymer particles.

As part of the dispersion, a RAFT agent functions as a stabiliser for the organic phase. Those skilled in the art will appreciate that the acronym “RAFT” stands for Reversible Addition Fragmentation chain Transfer, and that RAFT agents are used in a technique known as RAFT polymerisation.

RAFT polymerisation, as is described in International Patent Publication WO 98/01478, is a radical polymerisation technique that enables polymers to be prepared having a well defined molecular architecture and a narrow molecular weight distribution or low polydispersity.

RAFT polymerisation is believed to proceed under the control of a RAFT agent according to a mechanism which is simplistically illustrated below in Scheme 1.

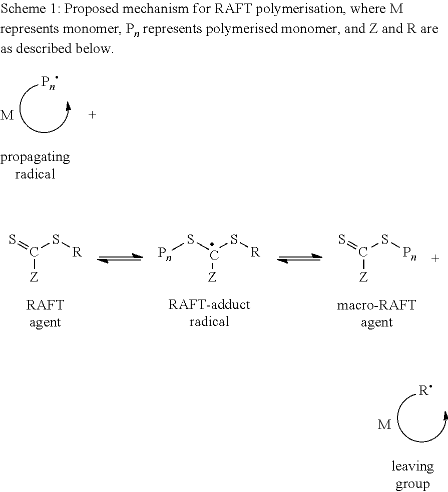

With reference to Scheme 1, R represents a group that functions as a free radical leaving group under the polymerisation conditions employed and yet, as a free radical leaving group, retains the ability to reinitiate polymerisation. Z represents a group that functions to convey a suitable reactivity to the C═S moiety in the RAFT agent towards free radical addition without slowing the rate of fragmentation of the RAFT-adduct radical to the extent that polymerisation is unduly retarded. Polymerisation of ethylenically unsaturated monomers using RAFT agents is well known to those skilled in the art.

As used herein, a “RAFT polymer” or a “RAFT polymer chain” is intended to mean a polymer/polymer chain that has been formed by a RAFT mediated polymerisation mechanism. A polymer chain comprising a RAFT agent may be referred to as a macro RAFT agent.

By the RAFT agent functioning as a “stabiliser” is meant that the agent serves to prevent, or at least minimise, coalescence or aggregation of the dispersed organic phase. As a stabiliser, the RAFT agent may prevent, or at least minimise, coalescence or aggregation of the organic phase through well known pathways such as steric and/or electrostatic repulsion. Those skilled in the art will appreciate that conventional surfactants have traditionally been employed in dispersions to perform such a function. The dispersion in accordance with the methods of the invention can advantageously be prepared without using conventional surfactants.

Where the dispersed organic phase is substantially in liquid form, the dispersion may in some technical fields be referred to as an emulsion. Upon polymerisation of the one or more ethylenically unsaturated monomers the dispersed organic phase will transition from being liquid into a solid, and the dispersion may instead be referred to as a colloidal suspension. Colloidal suspensions are often referred to in coating technology as emulsions, and the processes for preparing them referred to as emulsion polymerisations. An aqueous dispersion of polymer particles prepared by emulsion polymerisation may also be referred to as a “latex”.

Upon controlling the polymerisation of monomer, the RAFT agent, which may also be described as a macro RAFT agent, can also function to stabilise the so formed seed polymer particles in a similar manner to that outlined above in respect of the dispersed organic phase, again avoiding the need to use conventional surfactants. Such seed polymer particles may be described as being “self-stabilising” in the sense that conventional surfactants are not required to maintain them in a dispersed state.

To function as a stabiliser for the organic phase, RAFT agents used in accordance with the invention will typically have a structure that can form micelles in the continuous aqueous phase. Such agents will generally be amphipathic (i.e. comprise both hydrophilic and hydrophobic regions) and function as a stabiliser by virtue of a hydrophilic region associating with the aqueous phase and a hydrophobic region associating with the organic phase.

Compounds that function as a RAFT agent may not inherently also have an ability to function as a stabiliser in the context of the present invention.

RAFT agents that can function as a stabiliser in accordance with the invention, include those of general formula (II):

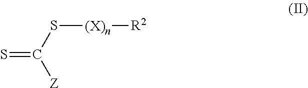

where each X is independently a polymerised residue of an ethylenically unsaturated monomer, n is an integer ranging from 0 to 100, for example from 1 to 60, or from 5 to 30, and R² and Z are each groups independently selected such that the agent can function as a RAFT agent in the polymerisation of the one or more ethylenically unsaturated monomers.

In order to function as a RAFT agent in the polymerisation of the one or more ethylenically unsaturated monomers, those skilled in the art will appreciate that R² will typically be an organic group which, in combination with the —(X)ₙ— group (i.e. as R¹—(X)ₙ—) if present, will function as a free radical leaving group under the polymerisation conditions employed while retaining the ability to reinitiate polymerisation. Similarly, those skilled in the art will appreciate that Z will typically be an organic group which functions to give a suitably high reactivity of the C═S moiety in the RAFT agent towards free radical addition without slowing the rate of fragmentation to the extent that polymerisation is unduly retarded.

Examples of suitable R² groups include alkyl, alkylaryl, arylalkyl, alkoxyaryl and alkoxyheteroaryl, each of which is optionally substituted with one or more hydrophilic groups.

More specific examples of suitable R² groups include C₁-C₆ alkyl, C₁-C₆ alkyl-C₆-C₁₈ aryl, C₆-C₁₈ aryl-C₁-C₆ alkyl, C₁-C₆ alkoxy-C₆-C₁₈ aryl and C₁-C₆ alkoxy-C₆-C₁₈ heteroaryl, each of which is optionally substituted with one or more hydrophilic groups.

Examples of hydrophilic substituent groups for R² may be selected from —CO₂H, —CO₂RN, —SO₃H, —OSO₃H, —SOR³N, —SO₂R³N, —OP(OH)₂, —P(OH)₂, —PO(OH)₂, —OH, —OR³N, —(OCH₂—CHR³)_{w}—OH, —CONH₂, CONHR′, CONR′R″, —NR′R″, —N⁺R′R″R″′, where R³ is selected from C₁-C₆ alkyl, w is 1 to 10, R′, R″ and R′″ are independently selected from alkyl and aryl which are optionally substituted with one or more hydrophilic substituents selected from —CO₂H, —SO₃H, —OSO₃H, —OH, —(COCH₂CHR³)_{w}—OH, —CONH₂, —SOR³ and SO₂R³, and salts thereof.

Still more specific examples of R² groups include —CH(CH₃)CO₂H, —CH(CO₂H)CH₂CO₂H, —C(CH₃)₂CO₂H and —CH₂Ph.

Specific examples of Z include optionally substituted alkoxy, optionally substituted aryloxy, optionally substituted alkyl, optionally substituted aryl, optionally substituted heterocyclyl, optionally substituted arylalkyl, optionally substituted alkylaryl, optionally substituted alkylthio, optionally substituted arylalkylthio, dialkoxy- or diaryloxy-phosphinyl [—P(═O)OR⁴₂], dialkyl- or diaryl-phosphinyl [—P(═O)R⁴₂], optionally substituted acylamino, optionally substituted acylimino, optionally substituted amino, R²—(X)ₙ—S— and a polymer chain formed by any mechanism; wherein R²; X and n are as defined above and R⁴ is selected from optionally substituted C₁-C₁₈ alkyl, optionally substituted C₂-C₁₈ alkenyl, optionally substituted aryl, optionally substituted heterocyclyl, optionally substituted aralkyl, optionally substituted alkaryl.

More specific examples of Z include —CH₂(C₆H₅), C₁-C₂₀ alkyl,

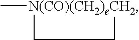

where e is 2 to 4, and —SR⁵, where R⁵ is selected from C₁ to C₂₀ alkyl.

Specific examples of optional substituents for R² and Z groups include epoxy, hydroxy, alkoxy, acyl, acyloxy, carboxy (and salts), sulfonic acid (and salts), alkoxy- or aryloxy-carbonyl, isocyanato, cyano, silyl, halo, and dialkylamino.

When the hydrophilic group of R² is —N⁺R′R″R″′, there will of course be an associated counter anion.

Other suitable RAFT agents include those of formula (II) in which R² is an organic group optionally substituted with one or more hydrophobic groups. In that case, Z will typically be an organic group optionally substituted with one or more hydrophilic groups.

Specific examples of RAFT agents or formula (II) include the agents of formula (III-XII):

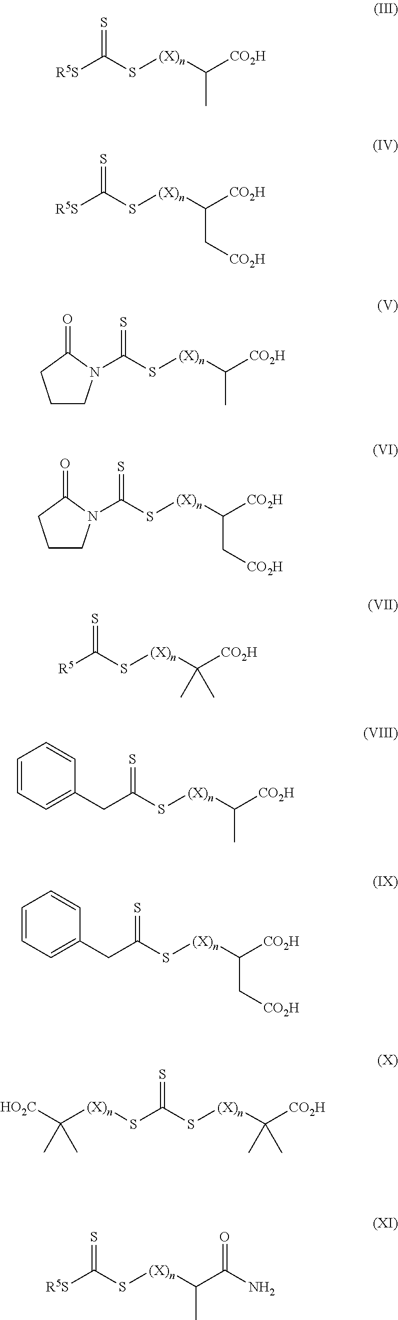

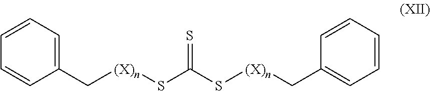

where X and n are as previously defined and R⁵ is selected from C₁ to C₂₀ alkyl.

It may be desirable that RAFT agents used in accordance with the invention demonstrate hydrolytic stability under the conditions of the polymerisation technique employed. Trithiocarbonyl RAFT agents have been found in general to exhibit hydrolytic stability.

As discussed above, the ability for RAFT agents used in accordance with the invention to function as a stabiliser will typically stem from its structure having hydrophilic and hydrophobic regions. Provided that the RAFT agent is capable of forming micelles in the aqueous phase, there is no particular limitation regarding the manner in which the structure of the RAFT agent presents the hydrophilic and hydrophobic regions. Examples of how a RAFT agent of formula (II) may present hydrophilic and hydrophobic regions include:
1) a combination of a hydrophobic end and a hydrophilic end; wherein the Z group provides hydrophobic properties to one end, and R² and —(X)ₙ— provide hydrophilic properties to the other end. In that case, —(X)ₙ— may be derived from hydrophilic monomer or be a tapered copolymer which gets progressively hydrophilic towards R²; or
2) a combination of a hydrophobic end and a hydrophilic end; wherein the Z group provides hydrophilic properties to one end, and R² and —(X)ₙ— provide hydrophobic properties to the other end. In that case, —(X)ₙ— may be derived from hydrophobic monomer or may be a tapered copolymer which gets progressively hydrophobic towards R²; or
3) a combination of a hydrophobic end and a hydrophilic end; wherein the Z group and —(X)ₙ— provide hydrophobic properties to one end, and R² provides hydrophilic properties to the other end; or
4) a combination of a hydrophobic end and a hydrophilic end; wherein the Z group provides hydrophobic properties to one end, —(X)ₙ— provides hydrophilic properties to the other end, and R² is hydrophobic such that the net effect of —(X)ₙ— and R² is to provide hydrophilic character to that end; or
5) a combination of hydrophilic ends and a hydrophobic middle section, wherein Z═—S—(X)ₙ—R², wherein each R² may be the same or different and provides hydrophilic properties to each end, and wherein —(X)ₙ— provides hydrophobic properties to the middle section; or
6) a combination of hydrophobic and hydrophilic properties within —(X)ₙ; wherein the portion of the —(X)ₙ— group closest to R² provides the hydrophilic properties and the portion of the —(X)ₙ— group closest to the thiocarbonylthio group provides the hydrophobic properties. In that case, —(X)ₙ— of formula (II) may be further represented as -(A)ₘ-(B)ₒ- to provide a block copolymer that has the following general formula (XIII):

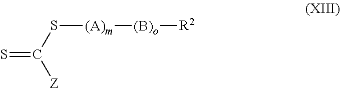

where formula (XIII) is a subset of formula (II) in which (X)ₙ is -(A)ₘ-(B)ₒ- and where each A and B is independently a polymerised residue of an ethylenically unsaturated monomer such that -(A)ₘ- provides hydrophobic properties and -(B)ₒ- provides hydrophilic properties, and m and o are independent integers ranging from 1 to 99, for example from 1 to 50, or from 1 to 30, or from 1 to 15, and Z is as described above. Z may also be chosen such that its polarity combines with that of -(A)ₘ- to enhance the overall hydrophobic character to that end of the RAFT agent. In addition to the hydrophilic character provided by -(B)ₒ-, R² may also be hydrophilic and enhance the overall hydrophilic character to that end of the RAFT agent, or R² may be hydrophobic provided that the net effect of -(B)ₒ- and R² results in an overall hydrophilic character to that end of the RAFT agent; or
7) a combination of hydrophilic ends and a hydrophobic middle section, wherein Z, of general formula (XIII), is —S-(A)ₘ-(B)ₒ-R², where -(A)ₘ- and -(B)ₒ- are as defined above. Each R² may be the same or different and the combination of the first -(B)ₒ-R² provides overall hydrophilic properties to one end, and the combination of the second -(B)ₒ-R² provides an overall hydrophilic properties to the other end. The hydrophobic portion of this type of amphiphilic RAFT agent is derived from the -(A)ₘ- regions.

RAFT agents used in accordance with the invention are generally selected or prepared in situ such that their amphipathic character is tailored to suit the particular mode of polymerisation to be employed. For example, integers m and o defined in general formula (XIII) may be selected such that:
i) for conventional emulsion polymerisation, m ranges from 1 to 20, or from 1 to 15, or from 1 to 10 (being at lower values within these ranges for more hydrophobic monomers, and at higher values within these ranges for less hydrophobic monomers); o ranges from 1 to 30, or from 1 to 10 or from 1 to 5 if (B) is derived from an ionic monomer; and o ranges from 1 to 80, or from 1 to 40 or from 1 to 30 if (B) is derived from a non ionic monomer;
ii) for miniemulsion and suspension polymerisation, m is at least 1, or at least 5, or at least 10; o is as defined above for conventional emulsion polymerisation.

It will be appreciated that the discussion above on different ways in which the RAFT agents can derive amphipathic character is presented in the context of such amphipathic character enabling the agents to function as a stabiliser for the dispersed organic phase.

In considering a suitable RAFT agent for use in accordance with the invention, the group represented by R² in formula (II) may be selected such that it is either hydrophilic or hydrophobic in character. Due to R² being somewhat removed from the thiocarbonylthio group, its role in modifying the reactivity of the RAFT agent becomes limited as n increases. However, it is important that groups —(X)ₙ—R² (formula II) and -(A)ₘ-(B)ₒ-R² (formula XIII) are nevertheless free radical leaving groups that are capable of reinitiating polymerisation.

The selection of Z is typically more important with respect to providing the RAFT agent with the ability to gain control over the polymerisation. In selecting a Z group it is generally important that such a group does not provide a leaving group that is a better leaving group in comparison with the —(X)ₙ—R² (formula II) or -(A)ₘ-(B)ₒ-R² (formula XIII) groups. By this limitation, monomer insertion preferentially occurs between —(X)ₙ—R² or -(A)ₘ-(B)ₙ-R² and its nearest sulphur atom.

In accordance with the methods of the invention, the one or more ethylenically unsaturated monomers are polymerised under the control of the RAFT agent. By being polymerised “under the control” of the RAFT agent is meant that polymerisation of the monomers proceeds via a reversible addition-fragmentation chain transfer (RAFT) mechanism to form polymer. Polymers prepared by RAFT polymerisation will typically have a lower polydispersity compared with the polymerisation being conducted in the absence of the RAFT agent.

The polymerisation will usually require initiation from a source of free radicals. The source of initiating radicals can be provided by any suitable method of generating free radicals, such as the thermally induced homolytic scission of suitable compound(s) (thermal initiators such as peroxides, peroxyesters, or azo compounds), the spontaneous generation from monomers (e.g. styrene), redox initiating systems, photochemical initiating systems or high energy radiation such as electron beam, X- or gamma-radiation. The initiating system is chosen such that under the reaction conditions there is no substantial adverse interaction of the initiator or the initiating radicals with the RAFT agent under the conditions of the reaction. The initiator ideally should also have the requisite solubility in the reaction medium.

Thermal initiators are chosen to have an appropriate half life at the temperature of polymerisation. These initiators can include one or more of the following compounds:
2,2′-azobis(isobutyronitrile), 2,2′-azobis(2-cyanobutane), dimethyl 2,2′-azobis(isobutyrate), 4,4′-azobis(4-cyanovaleric acid), 1,1′-azobis(cyclohexanecarbonitrile), 2-(t-butylazo)-2-cyanopropane, 2,2′-azobis {2-methyl-N-[1,1-bis(hydroxymethyl)-2-hydroxyethyl]propionamide}, 2,2′-azobis[2-methyl-N-(2-hydroxyethyl)propionamide], 2,2′-azobis(N,N′-dimethyleneisobutyramidine) dihydrochloride, 2,2′-azobis(2-amidinopropane) dihydrochloride, 2,2′-azobis(N,N-dimethyleneisobutyramidine), 2,2′-azobis{2-methyl-N-[1,1-bis(hydroxymethyl)-2-hydroxyethyl]propionamide}, 2,2′-azobis {2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, 2,2′-azobis(2,2,4-trimethylpentane), 2,2′-azobis(2-methylpropane), t-butyl peroxyacetate, t-butyl peroxybenzoate, t-butyl peroxyneodecanoate, t-butylperoxy isobutyrate, t-amyl peroxypivalate, t-butyl peroxypivalate, diisopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl hyponitrite, dicumyl hyponitrite. This list is not exhaustive.

Photochemical initiator systems are chosen to have the requisite solubility in the reaction medium and have an appropriate quantum yield for radical production under the conditions of the polymerisation. Examples include benzoin derivatives, benzophenone, acyl phosphine oxides, and photo-redox systems.

Redox initiator systems are chosen to have the requisite solubility in the reaction medium and have an appropriate rate of radical production under the conditions of the polymerisation; these initiating systems can include, but are not limited to, combinations of the following oxidants and reductants:
oxidants: potassium, peroxydisulfate, hydrogen peroxide, t-butyl hydroperoxide.
reductants: iron (II), titanium (III), potassium thiosulfite, potassium bisulfite.

Other suitable initiating systems are described in recent texts. See, for example, Moad and Solomon “the Chemistry of Free Radical Polymerisation”, Pergamon, London, 1995, pp 53-95.

Preferred initiating systems for conventional and mini-emulsion processes are those which are appreciably water soluble. Suitable water soluble initiators include, but are not limited to, 4,4-azobis(cyanovaleric acid), 2,2′-azobis{2-methyl-N-[1,1-bis(hydroxymethyl)-2-hydroxyethyl]propionamide}, 2,2′-azobis[2-methyl-N-(2-hydroxyethyl)propionamide], 2,2′-azobis(N,N′-dimethyleneisobutyramidine), 2,2′-azobis(N,N′-dimethyleneisobutyramidine) dihydrochloride, 2,2′-azobis(2-amidinopropane) dihydrochloride, 2,2′-azobis{2-methyl-N-[1,1-bis(hydroxymethyl)-2-ethyl]propionamide}, 2,2′-azobis[2-methyl-N-(2-hydroxyethyl)propionamide], 2,2′-azobis(isobutyramide) dihydrate, and derivatives thereof.

Preferred initiating systems for suspension polymerization are those which are appreciably soluble in the monomer to be polymerized. Suitable monomer soluble initiators may vary depending on the polarity of the monomer, but typically would include oil soluble initiators such as azo compounds exemplified by the well known material 2,2′-azobisisobutyronitrile. The other class of readily available compounds are the acyl peroxide class such as acetyl and benzoyl peroxide as well as alkyl peroxides such as cumyl and t-butyl peroxides. Hydroperoxides such as t-butyl and cumyl hydroperoxides are also widely used. A convenient method of initiation applicable to suspension processes is redox initiation where radical production occurs at more moderate temperatures. This can aid in maintaining stability of the polymer particles from heat induced aggregation processes.

During polymerisation of the one or more ethylenically unsaturated monomers to form the seed polymer particles, it is preferred that RAFT agent does not associate with or stabilise reservoir monomer droplets in the aqueous phase that ultimately are not destined to develop into polymer seed particles. Should this occur, control over the molecular weight and polydispersity of the resulting polymer that forms particles can be adversely affected.

A number of approaches can be adopted to minimise or avoid the RAFT agents associating with and stabilising reservoir monomer droplets, and these may vary depending on the polymerisation technique employed. For example, when conventional emulsion polymerisation is employed the RAFT agent that functions as the stabiliser may be prepared in situ. In that case, the dispersion of step (i) may be prepared by first forming a solution of a RAFT agent in the aqueous phase (i.e. where the agent is initially substantially soluble in the aqueous phase), and then adding ethylenically unsaturated monomer. Addition of monomer can be initially limited to minimise or avoid the formation of reservoir monomer droplets in the aqueous phase, while the agent is used to control the polymerisation of sufficient monomer such that it becomes substantially insoluble in the aqueous phase and in doing so self-assembles into micelles. Monomer in the aqueous phase can then migrate to the core of the micelles and thereby form the dispersed organic phase that is stabilised in the aqueous phase by the so formed stabilising RAFT agent. The monomers of the dispersed organic phase may then be polymerised under the control of the RAFT agent to form the seed polymer particles. Formation of the stabilising RAFT agent in situ and the subsequent polymerisation of the monomers to form the seed polymer particles may therefore be conducted as a continuos process.

It is believed that this approach in effect produces a stabilised organic phase whereby the RAFT agent is rendered non-labile. By rendering the RAFT agent substantially insoluble in the aqueous phase its potential to associate with and stabilise reservoir monomer droplets in the aqueous phase is believed to be significantly reduced. Once this point has been reached, further monomer can be added at a greater rate with little or no concern about forming stabilised reservoir monomer droplets in the aqueous phase. If and when additional RAFT agent is added during the polymerisation, the rate of addition of monomer may again be limited.

Where the methods of the inventions are performed using conventional emulsion polymerisation techniques, the polymerisation will typically be performed as a continuous or semi-continuous addition process rather than as a batch process. In particular, a batch process is more likely to result in a situation where RAFT agent can associate with or stabilise reservoir monomer droplets that ultimately will not develop into a polymer particle. If a batch process is to be used, it is preferable that the polymerisation proceed by miniemulsion or suspension techniques.

In the case of miniemulsion and suspension polymerisation, the dispersion of step (i) can be prepared by forming a composition comprising the ethylenically unsaturated monomer and RAFT agent that is substantially insoluble in the aqueous phase, and then combining this composition with the aqueous phase to form the dispersion. Generally, the RAFT agent in this case is substantially soluble in the monomer. Alternatively, the dispersion may be prepared by forming a composition comprising the aqueous phase and RAFT agent that is substantially insoluble in the aqueous phase, and then combining this composition with ethylenically unsaturated monomer. In either case, the RAFT agent used will at the outset be suitable to function as a stabiliser, or in other words the stabilising function of the agent will not be prepared in situ as described above.

By “combining this composition”, it is meant that the composition is combined so as to form the dispersion. Those skilled in the art will appreciate that once combined the resulting composition will generally also be subjected to a means for promoting the formation of a dispersion, such as applying shear to the combined composition. In the case of forming a composition comprising water insoluble RAFT agent and water, it may be necessary to subject this composition to means for forming a dispersion before the composition is combined with ethylenically unsaturated monomer.

In the case of miniemulsion and suspension polymerisation, sufficient RAFT agent is typically used to stabilise substantially all of the monomer present. By this approach, all monomer droplets should become polymer particles and reservoir monomer droplets are substantially avoided. Accordingly, in contrast to a conventional emulsion, it is preferable that these techniques are performed as a batch process. Where the polymerisation is carried out as a batch process, it is preferable that the RAFT agent used is substantially insoluble in the aqueous phase.

Having said this, a miniemulsion polymerisation performed initially as a batch process can be subsequently adapted to proceed as a continuous addition process through addition of further monomer and RAFT agent. Under these circumstances, RAFT agent that is soluble in the aqueous phase can be used provided that its addition occurs at such a time where substantially all of the monomer present is either dissolved in the water phase or solvated in polymer that has been formed. Once that state has been achieved, further monomer and RAFT agent can be added to the reaction system. In that case, monomer should nevertheless be added at such rate to avoid formation of reservoir monomer droplets while there is RAFT agent present that is still soluble in or can migrate through the aqueous phase (i.e. has not been rendered substantially insoluble in the aqueous phase as described above).

Where monomer is introduced throughout the polymerisation (e.g. in a continuous process), although not essential it may be desirable to also introduce an appropriate amount of conventional surfactant to assist with minimising or preventing the forming or formed seed particles associating with the interface that forms between the introduced monomer and the continuous aqueous phase.

The dispersion will generally be prepared using some form of agitation, for example shearing means. Techniques and equipment for this are well known in the art.

Having formed the dispersion, the one or more ethylenically unsaturated monomers are polymerised under the control of the RAFT agent to form the seed polymer particles. Where miniemulsion or suspension polymerisation techniques are used, the size of the resulting seed polymer particles are primarily dictated by the size of the dispersed organic phase droplets. Where conventional emulsion polymerisation techniques are used, the size of the resulting seed polymer particles are to a lesser extent dictated by the size of the dispersed organic phase droplets, and the amount of monomer introduced and subsequently polymerised is typically more determinative. Techniques for controlling the size of polymer particles formed by such dispersion polymerisation are well known in the art.

According to the present invention, both large and small seed polymer particles can be advantageously be prepared. The invention is particularly suited to preparing relatively small seed polymer particles. Without wishing to be limited by theory, it is believed that the dual function of the RAFT agent acting as an efficient stabiliser and also as a means for controlling the polymerisation of monomers assists with being able to prepare this diverse range in particle sizes.

In some embodiments, the largest dimension of the seed polymer particles will be no more than about one micron, for example no more than about 500 nm, no more than about 100 nm, no more than about 70 nm, no more than about 50 nm, no more than about 40 nm, and even no more than about 20 nm. The size of the final polymer particles will of corse depend on the amount of monomer that is expelled and subsequently polymerised on the surface of the seed particle.

The methods of the invention include crosslinking the seed polymer particles to form crosslinked seed polymer particles. By “crosslinking” is meant a reaction involving sites or groups on existing polymer chains or an interaction between existing polymer chains that results in the formation of at least a small region in the polymer chains from which at least four chains emanate.

The crosslinked seed polymer particles may be formed by any suitable means. Crosslinking may take place during formation of the seed polymer particles (i.e. as part of the polymerisation process), the seed particles may be formed and then subsequently crosslinked, or a combination of such techniques may be employed.

Those skilled in the art will appreciate that crosslinking may be achieved in numerous ways. For example, crosslinking may be achieved using multi-ethylenically unsaturated monomers. In that case, crosslinking is typically derived through a free radical reaction mechanism.

Alternatively, crosslinking may be achieved using ethylenically unsaturated monomers which also contain a reactive functional group that is not susceptible to taking part in free radical reactions (i.e. “functionalised” unsaturated monomers). In that case, such monomers may be incorporated into the polymer backbone through polymerisation of the unsaturated group, and the resulting pendant functional group provides means through which crosslinking may occur. By utilising monomers that provide complementary pairs of reactive functional groups (i.e. groups that will react with each other), the pairs of reactive functional groups can react through non-radical reaction mechanisms to provide crosslinks.

A variation on using complementary pairs of reactive functional groups is where the monomers are provided with non-complementary reactive functional groups. In that case, the functional groups will not react with each other but instead provide sites which can subsequently be reacted with a crosslinking agent to form the crosslinks. It will be appreciated that such crosslinking agents will be used in an amount to react with substantially all of the non-complementary reactive functional groups. Formation of the crosslinks under these circumstances will generally occur after polymerisation of the monomers. For example, seed particles may be formed where the polymer chains are provided with non-complementary groups, a crosslinking agent, capable of transfer through the aqueous phase, may then be added to the dispersion to diffuse into the particles and crosslink the polymer chains. In order to facilitate the diffusion of the cross linking agent into the particles it may prove useful to plasticise the particles with a small amount of monomer prior to adding the cross linking agent. Further swelling of the particles with monomer in accordance with the invention can then be achieved after the crosslinking reaction has been completed.

A combination of these crosslinking techniques may be used.

The terms “multi-ethylenically unsaturated monomers” and “functionalised unsaturated monomers” mentioned above can conveniently and collectively also be referred to herein as “crosslinking ethylenically unsaturated monomers” or “crosslinking monomers”. By the general term “crosslinking ethylenically unsaturated monomers” or “crosslinking monomers” it is meant an ethylenically unsaturated monomer through which a crosslink is or will be derived.

It will be appreciated that not all unsaturated monomers that contain a functional group will be used in accordance with the invention for the purpose of functioning as a crosslinking monomer. For example, acrylic acid should not be considered as a crosslinking monomer unless it is used to provide a site through which a crosslink is to be derived.

Examples of multi-ethylenically unsaturated monomers that may be used in accordance with the invention include ethylene glycol di(meth)acrylate, triethylene glycol di(meth)acrylate, tetraethylene glycol di(meth)acrylate, 1,3-butylene glycol di(meth)acrylate, trimethylolpropane tri(meth)acrylate, 1,4-butanediol di(meth)acrylate, neopentyl glycol di(meth)acrylate, 1,6-hexanediol di(meth)acrylate, pentaerythritol di(meth)acrylate, pentaerythritol tri(meth)acrylate, pentaerythritol tetra(meth)acrylate, glycerol di(meth)acrylate, glycerol allyloxy di(meth)acrylate, 1,1,1-tris(hydroxymethyl)ethane di(meth)acrylate, 1,1,1-tris(hydroxymethyl)ethane tri(meth)acrylate, 1,1,1-tris(hydroxymethyl)propane di(meth)acrylate, 1,1,1-tris(hydroxymethyl)propane tri(meth)acrylate, triallyl cyanurate, triallyl isocyanurate, triallyl trimellitate, diallyl phthalate, diallyl terephthalte, divinyl benzene, methylol (meth)acrylamide, triallylamine, oleyl maleate, glyceryl propoxy triacrylate, allyl methacrylate, methacrylic anhydride and methylenebis(meth) acrylamide.

Examples of ethylenically unsaturated monomers which contain a reactive functional group that is not susceptible to taking part in free radical reactions include acetoacetoxyethyl methacrylate, glycidyl methacrylate, N-methylolacrylamide, (isobutoxymethyl)acrylamide, hydroxyethyl acrylate, t-butyl-carbodiimidoethyl methacrylate, acrylic acid, γ-methacryloxypropyltriisopropoxysilane, 2-isocyanoethyl methacrylate and diacetone acrylamide.

Examples of pairs of monomers mentioned directly above that provide complementary reactive functional groups include N-methylolacrylamide and itself, (isobutoxymethyl)acrylamide and itself, γ-methacryloxypropyltriisopropoxysilane and itself, 2-isocyanoethyl methacrylate and hydroxyethyl acrylate, and t-butyl-carbodiimidoethyl methacrylate and acrylic acid.

Examples of crosslinking agents that can react with the reactive functional groups of one or more of the functionalised unsaturated monomers mentioned above include hexamethylene diamine, melamine, trimethylolpropane tris(2-methyl-1-aziridine propionate) and adipic bishydrazide. Examples of pairs of crosslinking agents and functionalised unsaturated monomers that provide complementary reactive groups include hexamethylene diamine and acetoacetoxyethyl methacrylate, hexamethylene diamine and glycidyl methacrylate, melamine and hydroxyethyl acrylate, trimethylolpropane tris(2-methyl-1-aziridine propionate) and acrylic acid, adipic bishydrazide and diacetone acrylamide.

Depending upon the manner in which crosslinking is achieved, it will be appreciated that the one or more ethylenically unsaturated monomers that are polymerised to form the seed polymer particles may comprise a mixture of non-crosslinking and crosslinking monomers. Alternatively, seed polymer particles may be formed from non-crosslinking monomers and subsequently swollen with crosslinking monomers that are in turn reacted to form the crosslinked seed polymer particles. In forming the crosslinked seed polymer particles, the crosslinking monomers will generally also be polymerised under the control of the RAFT agent.

When the seed polymer particles are prepared using batch polymerisation techniques such as miniemulsion polymerisation, the one or more ethylenically unsaturated monomers that are polymerised to form the seed polymer particles will generally comprise a mixture of non-crosslinking and crosslinking monomers.

RAFT agents of formula (II) may be prepared by numerous methods. Preferably the agents are prepared by polymerising ethylenically unsaturated monomers under the control of a RAFT agent of formula (XIV).

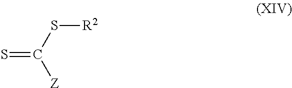

where Z and R² are as previously defined.

Specific examples of RAFT agents of formula (XIV) include the agents of formula (XV-XXIV):

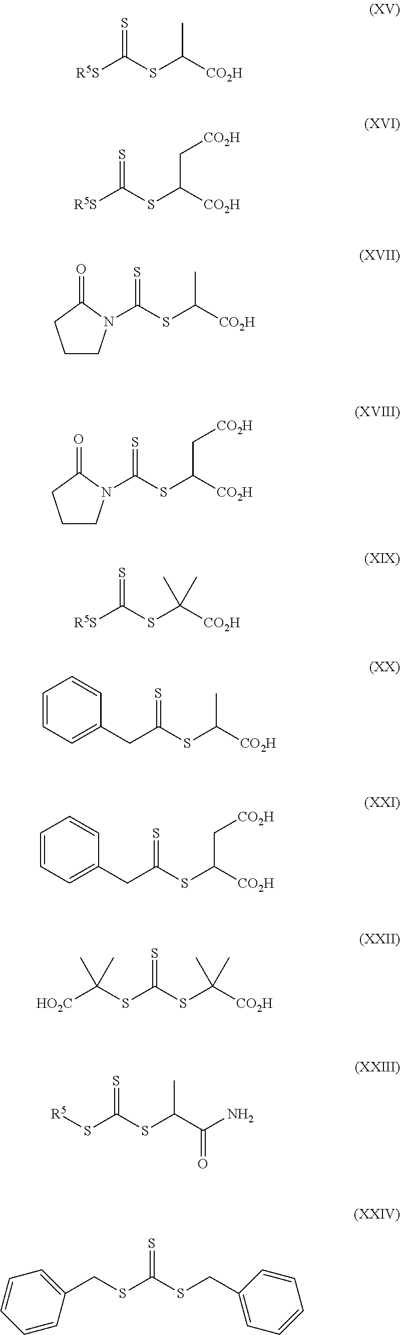

wherein R⁵ is as previously defined.

RAFT agents of formula (II) prepared from RAFT agents of formula (XIV) will have a structure that can stabilise the organic phase of the dispersion (typically exhibited by the agents ability to form micelles in the aqueous phase). Agents of formula (XIV) may inherently have some amphipathic character, however this will generally be insufficient to stabilise the organic phase of the dispersion. In order to achieve adequate stabilising properties, as in the context of agents of formula (II), agents of formula (XIV) will typically need to undergo reaction with suitable ethylenically unsaturated monomers.

Having said this, when n=0 in connection with formula (II), it is to be understood that such an agent will be inherently capable of stabilising the organic phase. In that case, formula (II) is equivalent to formula (XIV), and R² and Z in formula (XIV) are considered to provide adequate hydrophilic and hydrophobic properties in their own right to afford the required stabilising function.

Ethylenically unsaturated monomers that may be used to prepare RAFT agents of formula (II) may be selected from those monomers described herein above. To provide the so formed RAFT agent with sufficient amphipathic character to stabilise the organic phase, such monomers will typically be selected for their hydrophilic or hydrophobic qualities.

Examples of hydrophilic ethylenically unsaturated monomers include acrylic acid, methacrylic acid, hydroxyethyl methacrylate, hydroxypropyl methacrylate, acrylamide and methacrylamide, hydroxyethyl acrylate, N-methylacrylamide, dimethylaminoethyl methacrylate or other monomers that give a water soluble polymer directly or by suitable post reaction.

Examples of hydrophobic ethylenically unsaturated monomers include vinyl acetate, methyl methacrylate, methyl acrylate, styrene, alpha-methylstyrene, butyl acrylate, butyl methacrylate, amyl methacrylate, hexyl methacrylate, lauryl methacrylate, stearyl methacrylate, ethylhexyl methacrylate, crotyl methacrylate, cinnamyl methacrylate, oleyl methacrylate, ricinoleyl methacrylate, vinyl butyrate, vinyl tert-butyrate, vinyl stearate, vinyl laurate or other monomers that give a water insoluble polymer directly or by suitable post reaction.

The polymerisation of monomers to prepare RAFT agents of formula (II) may be conducted in either an aqueous solution or an organic solvent, the choice of which is dictated primarily by the nature of the monomers to be polymerised. Polymerisation may also be conducted in the monomer itself. The polymerisation reaction will usually require initiation from a source of radicals and these may be derived from the initiating systems described herein. Such initiators may be soluble in the monomer or monomer mixture.

One method for preparing a RAFT agent of formula (II), wherein R² has hydrophilic character, may comprise first selecting a suitable RAFT agent of formula (XIV). The selected RAFT agent is combined with a thermal initiator, solvent and hydrophilic monomer within a reaction vessel. Typically all reagents used are essentially free of dissolved oxygen and the reaction solution is purged of any remaining oxygen by way of an inert gas, such as nitrogen, prior to polymerisation. The reaction is subsequently initiated by increasing the temperature of the solution such that thermally induced homolytic scission of the initiator occurs. The polymerisation reaction then proceeds under control of the RAFT agent, thereby providing further hydrophilic character to the hydrophilic end of the RAFT agent through insertion of the hydrophilic monomer. For compounds of formula (II) in which Z is sufficiently hydrophobic, polymerisation of a second monomer may not be required. For compounds of formula (II) where Z is not sufficiently hydrophobic or for compounds of formula (XIII), upon exhaustion of the hydrophilic monomer, hydrophobic monomer may be added to the solution immediately, or at a later stage if the intermediate product is isolated, and the polymerisation continued under RAFT control to provide the block copolymer of formula (XIII). Where R² is intended to provide the hydrophobic properties to the RAFT agent, those skilled in the art will appreciate that the above method could be equally applied to prepare the “reverse” agent.

When compounds of formula (II) are prepared in accordance with the method described directly above and water is used as the solvent, upon reaching a point in the reaction where sufficient hydrophobic monomer has been polymerised by the RAFT agent, the propagating RAFT agent is believed to attain the requisite amphipathic character and subsequently self-assemble to form non-labile micelles, thus effectively preparing the stabilising RAFT agent in situ.

RAFT agents that self-assemble to form non-labile micelles can maintain their RAFT activity allowing polymerisation to continue under RAFT control within the hydrophobic core of the micelle. By this process, polymerisation may be continued as outlined above by supplying further monomer to prepare the aqueous dispersion of polymer seed particles. Crosslinking monomer may be included in this further monomer or crosslinking monomer may be subsequently introduced to afford the crosslinked seed polymer particles.

There are of course other technical variations on the way in which the method of preparing RAFT agents of formula (II) described above may be performed. For example, a RAFT agent may first undergo partial polymerisation with particular monomers so as not to be substantially amphipathic in character (e.g. to provide a RAFT agent that is substantially hydrophilic in character). This RAFT agent can then be isolated, and possibly stored, before use as an intermediate RAFT agent in subsequent preparation of the stabilising RAFT agent. Accordingly, a hydrophobic region may be subsequently added to the hydrophilic RAFT agent in a secondary reaction or during the course of an emulsion polymerisation to provide the diblock structure of formula (XIII). Alternatively, it may also be desirable to add a number of hydrophobic monomer units to a substantially hydrophilic RAFT agent prior to its isolation as an intermediate RAFT agent. Depending on the polarity of such a RAFT agent, subsequent use of it in an emulsion polymerisation reaction or water based secondary reaction may require a water miscible co-solvent to assist it in becoming properly dispersed. The intermediate RAFT agent may then be used in preparing the stabilising RAFT agent for use in accordance with the methods of the invention.

Throughout and upon their formation, the crosslinked seed polymer particles are advantageously stabilised by the now “polymerised” agent or macro RAFT agent. In other words, the hydrophilic region of the agent remains extended into the aqueous phase to prevent, or at least minimise, coalescence or aggregation of the particles and the hydrophobic region of the agent in effect forms part of the particle.

Having formed the crosslinked seed polymer particles, the methods of the invention comprise swelling the crosslinked seed particles with one or more ethylenically unsaturated monomers to form an aqueous dispersion of monomer swollen crosslinked seed polymer particles. Swelling of seed polymer particles with monomer in dispersion polymerisation techniques is a process well known to those skilled in the art. In general, the process involves introducing monomer to the aqueous dispersion such that the monomer is absorbed within the particles. It will be appreciated that such monomer will typically be substantially insoluble in the continuous aqueous phase and preferentially absorbed by the particles. The type of monomers employed for this purpose will therefore vary depending upon the composition of the aqueous phase and the nature of the polymer composition that forms the polymer particles. The one or more ethylenically unsaturated monomers that may be used to swell the crosslinked seed polymer particles include those described herein. Such monomers will generally be selected such that they give rise to a polymer on the surface of the seed particles that has a different molecular composition to that of the seed particles.

Provided that monomer can be expelled onto the surface of the crosslinked seed particles there is no particular limitation on the amount of monomer that is to be taken up by the particles. The particles are typically saturated with the selected monomer at room temperature (e.g. about 25° C.).

The methods in accordance with the invention also include increasing the temperature of the monomer swollen crosslinked seed polymer particles to expel at least some of the monomer therein onto the surface of the particles. Without wishing to be limited by theory, it is believed that upon heating the seed particle it's crosslinked structure contracts thereby expelling monomer from the particle.

The temperature increase of the seed particles required to promote expulsion of the monomer will generally vary depending upon the nature of the monomer and the crosslinked seed polymer particle, and also the amount of absorbed monomer within the particle.

Generally, the temperature of the polymer particles will be increased by at least 20° C., for example, at least 40° C., or even at least 60° C., relative to the temperature at which the particles were swollen with the monomer, to promote expulsion of the absorbed monomer.

In accordance with the methods of the invention, at least some of the monomer within the monomer swollen crosslinked seed polymer particles is expelled onto the surface of the particles. By the monomer being expelled “onto the surface of the particles” is meant that monomer coats at least some of the particle surface.

By controlling the manner in which the monomer is expelled onto the surface of the crosslinked seed polymer particles, polymer particles having different morphology can advantageously be prepared.

In one embodiment of the invention, monomer is expelled onto substantially the entire surface of the particles, or in other words is expelled such that the monomer substantially coats the entire particle surface. In that case, the monomer coated particles may be used to prepare core-shell polymer particles.

In a further embodiment, monomer is expelled onto only a proportion of the particle surface, or in other words only a proportion of the particle surface is coated with monomer. In that case, the partially monomer coated monomer particles may be used to prepared non-core-shell polymer particles.

In preparing such non-core-shell polymer particles, monomer will generally be expelled onto no more than about 70% of the particle surface, or no more than about 60% of the particle surface, or no more than about 50% of the particle surface. In some embodiments, the non-core-shell polymer particles are prepared by expelling at least some of the monomer onto only about 20% to about 60% of the particle surface. The proportion of the particle surface covered by the expelled monomer in the context of preparing the non-core-shell polymer particles will generally be that of a continuous surface coating and not that made up from two or more discontinuous surface coatings.

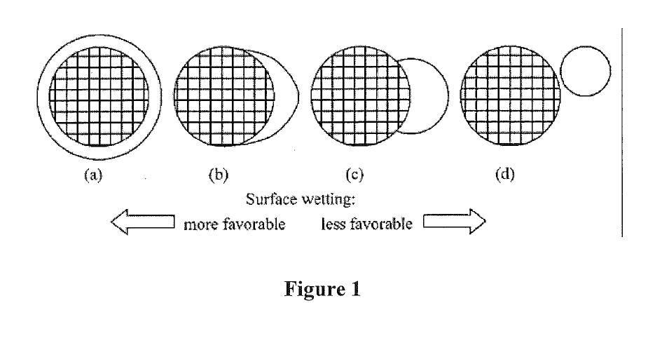

Without wishing to be limited by theory, the manner in which monomer is expelled onto the surface of the crosslinked seed polymer particles is believed to be at least in part controlled by monomer/polymer surface wetting properties. Thus, where the surface wetting properties of the crosslinked seed polymer particles are very favourable with the expelled monomer, the expelled monomer will tend to coat substantially the entire surface of the particle, whereas where such surface wetting is not at all favourable the expelled monomer may separate entirely from the surface of the particles. This is further illustrated in FIG. 1.

With reference to FIG. 1, it will be appreciated that (d) represents unfavourable surface wetting between the expelled monomer and the surface of the crosslinked seed polymer particle and therefore does not fall within the scope of the present invention (i.e. the expelled monomer does not form on the surface of the particle). In contrast, the present invention requires at least some monomer to be expelled onto the surface of the particles (i.e. as in illustrations (a), (b) and (c)). Thus, core-shell polymer structures may be prepared when the surface wetting between the expelled monomer and surface of the crosslinked seed polymer particles is very favourable (i.e. illustration (a)), and non-core-shell polymer structures may be prepared when the surface wetting properties between the expelled monomer and the surface of the crosslinked seed polymer particle is mildly or not very favourable (i.e. illustrations (b) to (c)).

Having regard to at least surface wetting characteristics, a person skilled in the art will be able to select a suitable combination of crosslinked seed polymer particles and monomer(s) to be absorbed therein that will afford the desired degree of surface coating upon expelling monomer.

In general, the wetability of the surface of the crosslinked seed polymer particles with a given expelled monomer may be controlled by altering the way in which the crosslinked seed polymer particles are prepared. Thus, unfavourable wetting may occur where the expelled monomer is relatively hydrophobic and the surface of the crosslinked seed polymer particles is relatively hydrophilic. In that case, wetting can be rendered more favourable by increasing the hydrophobic character of the surface of the particles. Alternatively, wetting can be rendered more favourable by using monomer that is relatively or more hydrophilic.

Those skilled in the art will appreciate that by virtue of the manner in which the crosslinked seed polymer particles are prepared, their surface comprises a relatively hydrophilic region or segment of the RAFT agent that associates with the continuous aqueous phase and functions to stabilise the particles. The nature of this stabilising region or segment of the RAFT agent can therefore be manipulated to modify the hydrophilic, and thus wetability, properties of the particle surface. For example, a relatively hydrophobic expelled monomer is likely to more readily wet the surface of the particles when the hydrophilic region or segment of the RAFT agent is derived from 2-hydroxy ethyl acrylate compared with acrylamide monomer. Also, where the stabilising hydrophilic region or segment of the RAFT agent is derived from acrylic acid monomer, the surface of the particle is likely to be more readily wet by a relatively hydrophobic expelled monomer when the carboxylic acid moieties of the polyacrylic acid region or segment are not ionised compared with when they are ionised. In that case, adjustment of the pH of the continuous aqueous phase may be used to manipulate the surface wetting characteristics of the seed particles.

By virtue of the manner in which the crosslinked seed polymer particles are prepared, those skilled in the art will also appreciate that initiator residues used to initiate polymerisation of the one or more ethylenically unsaturated monomers may also form part of the hydrophilic region or segment of the RAFT agent that associates with the continuous aqueous phase and functions to stabilise the particles. The hydrophilic character of the surface of the particles may therefore be altered by using different initiators. For example, initiators that provide for moieties comprising persulfate or carboxylic acid groups may be employed and the hydrophilic character of the particle surface modified via pH adjustment as described above.

A combination of such approaches may of course be used in order to modify the hydrophilic character of the particle surface.

In addition to, or separate from, modifying the hydrophilic character of the particle surface to adjust wetting, the nature of the monomer to be absorbed within and subsequently expelled onto the surface of the particles may be selected to be relatively more or less hydrophobic compared with the surface of the particles. For example, a less hydrophobic monomer such as methyl methacrylate may be selected in preference to a more hydrophobic monomer such as styrene.

As will be appreciated from the discussion above, the surface wetting properties of the crosslinked seed polymer particles will be determined to a large extent by the nature of the relatively hydrophilic region or segment of the RAFT agents that associate with the continuous aqueous phase and function to stabilise the particles. As monomer is expelled onto the surface of the particles, some or all of such stabilising moieties may be partially or entirely engulfed by the expelled monomer and thereby render the stabilising moieties ineffective. Under these circumstances, it may be necessary to introduce before or at the time when the monomer is expelled a stabiliser to assist with maintaining the particles in a dispersed state in the continuous aqueous phase. The type of stabilising agent employed may depend upon the nature of the composition of the dispersion and/or the temperature at which the subsequent polymerisation reaction is to be conducted. Those skilled in the art will be able to select a suitable stabilising agent for a given dispersion and reaction conditions.

Suitable stabilisers (i.e. conventional surfactants) include anionic surfactants such as dodecyl sulphate, nonyl phenol ethoxylate sulphates, alkyl ethoxylate sulphates, alkyl sulphonates, alkyl succinates, alkyl phosphates, alkyl carboxylates and other alternatives well known to those skilled in the art. Other suitable stabilising agents include polymeric stabilisers, cationic surfactants and non-ionic surfactants.

As discussed above in connection with the dispersed organic phase and the dispersed seed polymer particles, the RAFT agent can also function to stabilise the so formed polymer particles having core-shell or non-core-shell morphology, again advantageously avoiding the need to use conventional surfactants. Such polymer particles may also be described as being “self-stabilising” in the sense that conventional surfactants are not required to maintain them in a dispersed state.

The surface properties of polymer particles in accordance with the invention are important when it comes to preparing Janus polymer particles. In particular, the Janus polymer particles must present two faces or surfaces that each have a different molecular composition. As discussed above, the surface characteristics of polymer particles prepared in accordance with the invention can be influenced by the nature of the relatively hydrophilic stabilising region or segment of RAFT agents covalently bound to the surface of the crosslinked seed particles. Thus, Janus particle character can advantageously be derived where only a portion of monomer is expelled onto the surface of the seed particles such at least some of the hydrophilic stabilising region or segment of RAFT agents covalently bound to that portion of the crosslinked seed particles is engulfed. In that case, the particles can present one face or surface reflecting the character of the hydrophilic stabilising region or segment of RAFT agents and a second face or surface reflecting the character of the expelled monomer/polymer (i.e. the expelled monomer will be subsequently polymerised to form polymer).

Manipulation of the hydrophilic stabilising region or segment of the RAFT agents and/or the manner in which monomer is expelled onto the surface of the crosslinked seed particles can afford non-core-shell polymer particles in the form of Janus polymer particles.

Those skilled in the art will appreciate that formation of Janus character is more of a continuum rather than a discrete transition. Nevertheless, polymer particles according to the present invention are considered to exhibit Janus character or be Janus particles when they exhibit surface active properties.

When the monomer is expelled onto the surface of the particles, some of the absorbed monomer may remain within the particle (i.e. not be expelled).

Upon expelling monomer onto the surface of the particles, and if required introducing stabiliser to maintain the particles in a dispersed state, the expelled monomer is polymerised to form polymer on the surface of the particles. As there will usually be at least some monomer that still remains within the seed particle, the polymerisation may also extend within the seed particle thereby intermeshing the newly formed surface polymer with the existing crosslinked seed polymer particle.

The polymerisation reaction will usually require initiation from a source of radicals and these may be derived from the initiating systems described herein.

As outlined above, depending upon the amount of particle surface covered by the expelled monomer, the polymerisation may give rise to core-shell or non-core-shell polymer particles.

The methods in accordance with the invention are particularly well suited for preparing polymer particles having a largest dimension of no more than about 100 nm, of no more than about 70 nm, of no more than about 50 nm, and even of no more than about 40 nm.

The present invention can therefore be employed to prepare core-shell and non-core-shell polymer particles having a largest dimension of no more than about 100 nm, of no more than about 70 nm, of no more than about 50 nm, and of no more than about 40 nm.

The methods of the invention are believed to afford polymer particles that have a unique composition.

The polymer particles are capable of being dispersed a liquid. By being “capable” is meant that the particles have suitable properties (e.g. size, composition) that enable them to be dispersed in a liquid. In other words, the polymer particles are suitable for being dispersed a liquid.

In one embodiment, the polymer particles are self-stabilising in the sense that they can be dispersed in a liquid (e.g. an aqueous liquid) without using an introduced or separate stabiliser.

The particles comprise two polymer regions of different molecular composition, wherein one of the polymer regions is a crosslinked RAFT polymer having covalently bound to its surface RAFT polymer chains that function as a stabiliser when the particles are dispersed in the liquid. By functioning as a stabiliser the RAFT polymer chains serve to prevent, or at least minimise, coalescence or aggregation of the dispersed particles. Where the polymer particles are self-stabilising, there is sufficient stabilising effect provided by the RAFT polymer chains alone.

The crosslinked RAFT polymer region of the particles is as described in detail above relating to the method of the invention. The at least one other polymer region of the particles is as described in detail above for the “expelled monomer” that is polymerised into polymer relating to the method of the invention.

Such polymer particles may have core-shell or non-core-shell structures, and the non-core-shell structures may present as Janus particles.

Where the polymer particles have a core-shell structure, the crosslinked RAFT polymer region will represent the core of the structure.

As alluded to above, it will be appreciated that the RAFT polymer chains that function as a stabiliser are derived from the RAFT agent that stabilises the organic phase in accordance with the methods of the invention. The function of the RAFT polymer chains “as a stabiliser” is therefore similar to that discussed above in the context of the methods of the invention.

Stabilisation of the polymer particles in a liquid may be facilitated with one or more conventional surfactants as herein before described.

The polymer particles will generally have a composition such that they are capable of being dispersed in an aqueous liquid.

The morphology and size of the resulting particles may be assessed with analytical techniques well known to those skilled in the art. For example, the particles may be analysed using Transition Electron Microscopy (TEM).

The diverse array of polymer particle size and/or morphologies that can be formed in accordance with the invention are expected to give rise to new materials applications. For example, the polymer particles may find use in coatings (eg. paint), adhesive, filler, primer, sealant, pharmaceutical, cosmetic, diagnostic and therapeutic applications.

Polymer particle morphologies formed in accordance with the invention may in their own right give rise to unique properties. For example, anisotropic non-core-shell polymer particles prepared in accordance with the invention may give rise to unique asymmetric interactions.

It has also been found that polymer particles in accordance with the invention may be used as Pickering stabilisers in the manufacture of polymer particles. In particular, polymer particles in accordance with the invention may be employed in a conventional emulsion or miniemulsion process to stabilise a dispersed organic phase comprising one or more ethylenically unsaturated monomers. Monomer in the dispersed organic phase may then be polymerised to form polymer particles that can be used in a variety of applications.

In other words, polymer particles in accordance with the invention can advantageously be used in place of a surfactant to stabilise dispersed monomer when preparing polymer particles by a conventional emulsion or miniemulsion process.

The present invention therefore also provides a coatings, adhesive, filler, primer, sealant, pharmaceutical, cosmetic, diagnostic, and therapeutic product comprising polymer particles prepared in accordance with the invention.

As used herein, the term “alkyl”, used either alone or in compound words denotes straight chain, branched or cyclic alkyl, preferably C₁₋₂₀ alkyl, e.g. C₁₋₁₀ or C₁₋₆ Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo).

The term “aryl” (or “carboaryl”) denotes any of single, polynuclear, conjugated and fused residues of aromatic hydrocarbon ring systems, preferably C₆₋₂₄ (e.g. C₆₋₁₈ or C₆₋₁₂). Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl. A carbocyclyl group may be optionally substituted by one or more optional substituents as herein defined. The term “carbocyclylene” is intended to denote the divalent form of carbocyclyl.

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl. A heteroaryl group may be optionally substituted by one or more optional substituents as herein defined. The term “heteroarylene” is intended to denote the divalent form of heteroaryl.

The term “acyl” either alone or in compound words denotes a group containing the moiety C═O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{e} residue may be optionally substituted as described herein.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl, aryl, heteroaryl, heterocyclyl, carbocyclyl and aralkyl. Examples of preferred R^{f} include C₁₋₂₀alkyl, phenyl and benzyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In one embodiment at least one R^{f} is hydrogen. In another embodiment, both R^{f} are hydrogen.

The term, “amino” is used here in its broadest sense as understood in the art and includes groups of the formula NR^{a}R^{b} wherein R^{a} and R^{b} may be any independently selected from hydrogen, alkyl, alkenyl, alkynyl, aryl, carbocyclyl, heteroaryl, heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(O)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{a}R^{b}, wherein R^{a} and R^{b} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g. CO₂phenyl), CO₂aralkyl (e.g. CO₂ benzyl).

As used herein, the term “aryloxy” refers to an “aryl” group attached through an oxygen bridge. Examples of aryloxy substituents include phenoxy, biphenyloxy, naphthyloxy and the like.

As used herein, the term “acyloxy” refers to an “acyl” group wherein the “acyl” group is in turn attached through an oxygen atom. Examples of “acyloxy” include hexylcarbonyloxy (heptanoyloxy), cyclopentylcarbonyloxy, benzoyloxy, 4-chlorobenzoyloxy, decylcarbonyloxy (undecanoyloxy), propylcarbonyloxy (butanoyloxy), octylcarbonyloxy (nonanoyloxy), biphenylcarbonyloxy (eg 4-phenylbenzoyloxy), naphthylcarbonyloxy (eg 1-naphthoyloxy) and the like.

As used herein, the term “alkyloxycarbonyl” refers to a “alkyloxy” group attached through a carbonyl group. Examples of “alkyloxycarbonyl” groups include butylformate, sec-butylformate, hexylformate, octylformate, decylformate, cyclopentylformate and the like.

As used herein, the term “arylalkyl” refers to groups formed from straight or branched chain alkanes substituted with an aromatic ring. Examples of arylalkyl include phenylmethyl (benzyl), phenylethyl and phenylpropyl.

As used herein, the term “alkylaryl” refers to groups formed from aryl groups substituted with a straight chain or branched alkane. Examples of alkylaryl include methylphenyl and isopropylphenyl.

In this specification “optionally substituted” is taken to mean that a group may or may not be substituted or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate, phosphate, triarylmethyl, triarylamino, oxadiazole, and carbazole groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

Preferred optional substituents include alkyl, (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), nitro, formyl, —C(O)-alkyl (e.g. C₁₋₆ alkyl, such as acetyl), O—C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g. C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl)aminoalkyl (e.g., HN C₁₋₆ alkyl-, C₁₋₆alkylHN—C₁₋₆ alkyl- and (C₁₋₆ alkyl)₂N—C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆alkyl(O)CC₁₋₆alkyl-), nitroalkyl (e.g., O₂NC₁₋₆alkyl-), sulfoxidealkyl (e.g., R(O)SC₁₋₆ alkyl, such as C₁₋₆ alkyl(O)SC₁₋₆ alkyl-), sulfonylalkyl (e.g., R(O)₂SC₁₋₆ alkyl- such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-), triarylmethyl, triarylamino, oxadiazole, and carbazole.

The invention will now be described with reference to the following non-limiting examples.

### EXAMPLES

### Example 1

### Synthesis of Anisotropic Nanoparticles Using Diblock Poly(AA-b-Sty) of 2-{[(butylsulfanyl)carbonothioyl]sulfanyl}propanoic acid RAFT Agent

Part 1.1: Preparation of a Diblock Poly[(Styrene)ₘ-b-(Acrylic Acid)ₙ] Macro-RAFT Agent with Respective Degrees of Polymerization m≈30 and n≈20, in Dioxane

A solution of 2-{[(butylsulfanyl)carbonothioyl]sulfanyl}propanoic acid (0.801 g, 3.359 mmol), 4,4′-azobis(4-cyanovaleric acid) (0.049 g, 0.173 mmol), acrylic acid (4.84 g, 67.15 mmol) in dioxane (22.03 g) was prepared in a 100 mL round bottom flask. This was stirred magnetically and sparged with nitrogen for 20 minutes. The flask was then placed in an 80° C. oil bath for 2.5 hours with constant stirring. To the reacted mixture, styrene (10.25 g, 98.45 mmol), 4,4′-azobis(4-cyanovaleric acid) (0.05 g, 0.178 mmol) and dioxane (24.52 g) were added and again sparged with nitrogen for 10 minutes. The flask was then placed in an 80° C. oil bath for 2 hours with constant stirring. The same amount of initiator, 4,4′-azobis(4-cyanovaleric acid) (0.05 g, 0.178 mmol), was added four consecutive times after 2 hours reaction interval. The final copolymer solution had solids of 20%. The dioxane was then dried in a vacuum oven to yield a yellow powder.

### Part 1.2: Synthesis of Divinyl Benzene Crosslinked Polystyrene Nanoparticles Using the Macro-RAFT Agent Prepared in Part 1.1

Macro-RAFT agent from Part 1.1 (0.081 g), sodium hydroxide (0.025 g, 0.616 mmol) were dissolved in water (2.35 g) in a 20 mL flask; a clear solution was obtained after initial stirring on a magnetic stirrer was followed by sonication in a sonic bath for 30 minutes. To this solution styrene (0.254 g, 2.437 mmol) and water (8.03 g) were added. The mixture was stirred overnight. 4,4′-azobis(4-cyanovaleric acid) (0.01 g, 0.036 mmol) was added to the monomer swollen micelles. The flask was sealed and subsequently deoxygenated with nitrogen sparging for 10 minutes. The whole flask was immersed in an oil bath with a temperature setting of 80° C. and maintained at that temperature for 4 hours under constant magnetic stirring. Divinyl benzene (0.1 g, 0.77 mmol) and 2,2′-azobisisobutylronitrile (0.014 g, 0.085 mmol) were then added, stirring to mix for 2 hours at room temperature. The whole flask was immersed back in an oil bath with a temperature setting of 75° C. and maintained at that temperature for overnight under constant magnetic stirring. The resulting latex had average diameter of 17 nm by light scattering.

### Part 1.3: Synthesis of Polystyrene Anisotropic Particles Using the Crosslinked Polystyrene Seeds Prepared in Part 1.2

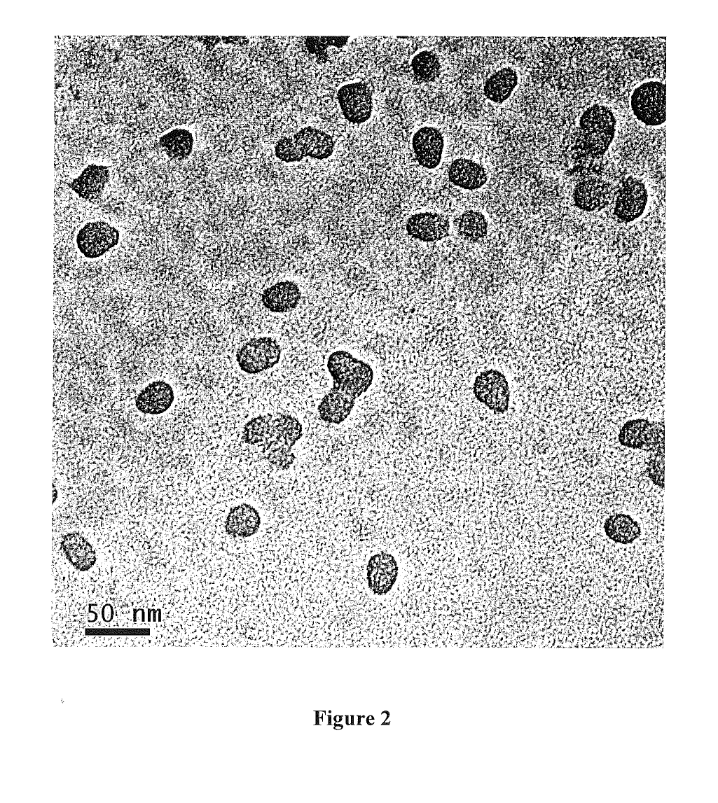

A mixture of the polystyrene latex from Part 1.2 (5.08 g), styrene (0.363 g, 3.488 mmol) and sodium dodecyl sulphate (0.027 g, 0.094 mmol) were prepared in a 20 mL flask and stirred overnight. 4,4′-azobis(4-cyanovaleric acid) (0.02 g, 0.071 mmol) was added to the monomer swollen latex particles. The flask was sealed and stirred at room temperature for 2 hours, subsequently deoxygenated with nitrogen sparging for 10 minutes. The whole flask was immersed in an oil bath with a temperature setting of 80° C. and maintained at that temperature for 4 hours under constant magnetic stirring. The resulting latex was analysed using Transmission Electron Microscopy (TEM). The TEM image (see FIG. 2) shows that the final latex comprised monodisperse football or anisotropic nanoparticles having average dimensions of 23 nm wide and 35 nm long. Both ends of the particle are formed from polystyrene, but the polystyrene end derived from the expelled monomer is not crosslinked.

### Example 2

### Synthesis of Anisotropic Nanoparticles Using Diblock Poly(AA-b-Sty) of 2-{[(butylsulfanyl)carbonothioyl]sulfanyl}propanoic acid RAFT Agent

### Part 2.1: Synthesis of Divinyl Benzene Crosslinked Polystyrene Nanoparticles Using the Macro-RAFT Agent Prepared in Part 1.1

A clear solution of macro-RAFT agent from Part 1.1 (0.379 g), sodium hydroxide (0.075 g, 1.864 mmol) and water (7.57 g) was prepared in a 20 mL flask, stirring on a magnetic stirrer, which was followed by sonication in a sonic bath for 30 minutes. To this solution styrene (0.855 g, 8.207 mmol) and sodium hydrogen carbonate (8.7 mg, 0.101 mmol) were added, stirring overnight. Potassium persulphate (0.02 g, 0.073 mmol) was added to the monomer swollen micelles. The flask was sealed and subsequently deoxygenated with nitrogen sparging for 10 minutes. The whole flask was immersed in an oil bath with a temperature setting of 80° C. and maintained at that temperature for 4 hours under constant magnetic stirring. Divinyl benzene (0.346 g) and 2,2′-azobisisobutylronitrile (0.032 g, 0.192 mmol) were then added, stirring to mix for 1 hour at room temperature. The whole flask was immersed back in an oil bath with a temperature setting of 75° C. and maintained at that temperature overnight under constant magnetic stirring. The final latex particles had average diameters of 27 nm by light scattering.

### Part 2.2: Synthesis of Anisotropic Nanoparticles Using the Crosslinked Polystyrene Seeds Prepared in Part 2.1

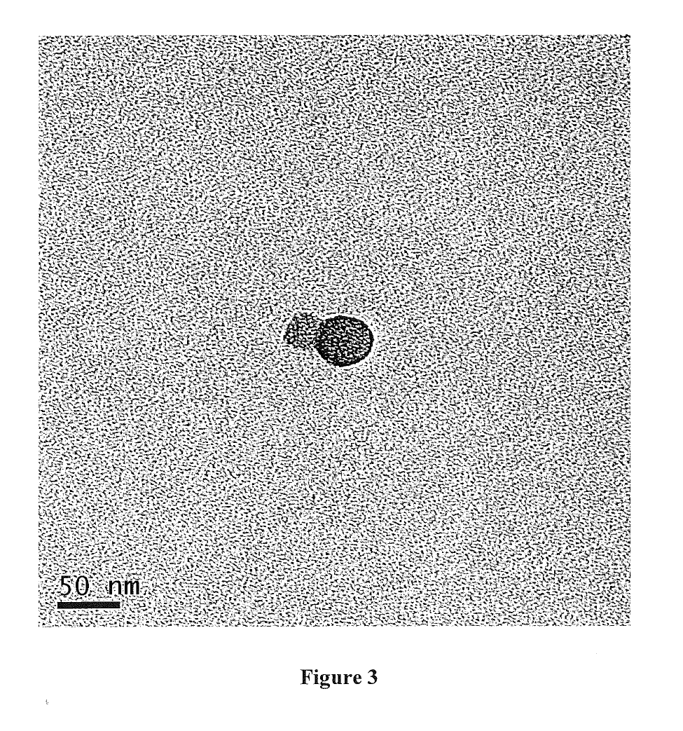

A mixture of the styrene latex from Part 2.1 (1.95 g), methyl methacrylate and butyl acrylate monomer mixture at the weight ratio of 7:3 (0.50 g) and water (4.68 g) were prepared in a 20 mL flask and stirred overnight. 2,2′-azobisisobutylronitrile (0.015 g, 0.089 mmol) was added to the monomer swollen latex particles. The flask was sealed and stirred at room temperature for 2 hours, subsequently deoxygenated with nitrogen sparging for 10 minutes. The whole flask was immersed in an oil bath with a temperature setting of 75° C. and maintained at that temperature for 4 hours under constant magnetic stirring. The resulting latex was analysed using TEM. The TEM image (see FIG. 3) showed that the final latex comprised monodisperse dumbbell shaped nanoparticles with each half having average diameter of 23 nm. One end of the particle is formed from polystyrene (i.e. the original crosslinked seed polymer particle) while the other end is derived from the expelled monomer and is formed of polymethyl methacrylate-co-butyl acrylate.

### Example 3

### Synthesis of Pickering Emulsion Particles Using Anisotropic Nanoparticles as Stabilisers

### Part 3.1a Synthesis of Pickering Polystyrene Latex Particles Using Anisotropic Particles Prepared in Part 1.3

A mixture of the styrene latex from Part 1.3 (1.05 g), styrene monomer (5.64 g), sodium hydroxide (0.12 g), 4,4′-azobis(4-cyanovaleric acid) (V-501, 0.09 g) and water (22.08 g) were prepared in a 50 mL flask. The flask was sealed, stirred at room and deoxygenated with nitrogen sparging for 10 minutes. The whole flask was immersed in an oil bath with a temperature setting of 70° C. and maintained at that temperature for 17 hours under constant magnetic stirring. Transmission electron microscopy showed that the final latex contained monodisperse particles with Z-average diameter of 215 nm and PDI of 0.045 by light scattering.

### Comparative Example

Part 3.1b: Synthesis of Surfactant Free Emulsion without Anisotropic Nanoparticles Prepared in Accordance with the Invention.

A mixture of styrene monomer (5.604 g), sodium hydroxide (0.095 g), 4,4′-azobis(4-cyanovaleric acid) (V-501, 0.094 g) and water (22.29 g) were prepared in a 50 mL flask. The flask was sealed, stirred at room and deoxygenated with nitrogen sparging for 10 minutes. The whole flask was immersed in an oil bath with a temperature setting of 70° C. and maintained at that temperature for 7 hours under constant magnetic stirring. Transmission electron microscopy showed that the final latex contained monodisperse particles with Z-average diameter of 610 nm and PDI of 0.207 by light scattering.

### Part 3.2 Synthesis of Pickering Poly(Methacrylates/Butyl Acrylate) Latex Particles Using Anisotropic Nanoparticles Prepared in Part 1.3

A mixture of the styrene latex from Part 1.3 (0.53 g), methyl methacrylate and butyl acrylate monomer mixture at the weight ratio of 1:1 (2.84 g), sodium hydroxide (0.12 g), 4,4′-azobis(4-cyanovaleric acid) (V-501, 0.09 g) and water (20.03 g) were prepared in a 50 mL flask. The flask was sealed, stirred at room and deoxygenated with nitrogen sparging for 10 minutes. The whole flask was immersed in an oil bath with a temperature setting of 70° C. and maintained at that temperature for 17 hours under constant magnetic stirring. Transmission electron microscopy showed that the final latex particles were clearly stabilised by the non-core-shell polystyrene nanoparticles. The final particles had Z-average diameter of 229 nm and PDI of 0.027 by light scattering.

### Part 3.3 Synthesis of Pickering Poly(Methacrylates/Butyl Acrylate) Latex Particles Using Anisotropic Nanoparticles Prepared in Part 1.3

A mixture of the styrene latex from Part 1.3 (1.01 g), methyl methacrylate and butyl acrylate monomer mixture at the weight ratio of 1:1 (1.13 g), sodium hydroxide (0.06 g), 4,4′-azobis(4-cyanovaleric acid) (V-501, 0.04 g) and water (10.06 g) were prepared in a 50 mL flask. The flask was sealed, stirred at room and deoxygenated with nitrogen sparging for 10 minutes. The whole flask was immersed in an oil bath with a temperature setting of 70° C. and maintained at that temperature for 17 hours under constant magnetic stirring. Transmission electron microscopy showed that the final latex particles were clearly stabilised by the non-core-shell polystyrene nanoparticles. The final particles had Z-average diameter of 147 nm and PDI of 0.008 by light scattering.

### Part 3.4 Synthesis of Pickering Polystyrene Latex Particles Using Anisotropic Nanoparticles Prepared in Part 2.2

A mixture of the styrene latex from Part 2.2 (0.30 g), styrene monomer (3.50 g), sodium hydroxide (0.13 g), 4,4′-azobis(4-cyanovaleric acid) (V-501, 0.10 g) and water (27.11 g) were prepared in a 50 mL flask. The flask was sealed, stirred at room and deoxygenated with nitrogen sparging for 10 minutes. The whole flask was immersed in an oil bath with a temperature setting of 70° C. and maintained at that temperature for 7 hours under constant magnetic stirring. Transmission electron microscopy showed that the final latex particles were clearly stabilised by the non-core-shell nanoparticles. The final particles had Z-average diameter of 189 nm and PDI of 0.018 by light scattering.

### Example 4

### Synthesis of Anisotropic Nanoparticles Using Diblock Poly(AAm-b-Sty) of 2-{[(butylsulfanyl)carbonothioyl]sulfanyl}propanoic acid RAFT Agent

Part 4.1: Preparation of a Diblock Poly[(Styrene)ₘ-b-(Acrylamide)ₙ] Macro-RAFT Agent with Respective Degrees of Polymerization m≈9 and n≈15, in Water/Dioxane Solvent

A solution of 2-{[(butylsulfanyl)carbonothioyl]sulfanyl}propanoic acid (0.801 g, 3.361 mmol), 4,4′-azobis(4-cyanovaleric acid) (0.100 g, 0.360 mmol), acrylamide (3.711 g, 52.213 mmol) in a solvent mixture of dioxane (6.61 g) and water (4.41 g) was prepared in a 50 mL round bottom flask. This was stirred magnetically and sparged with nitrogen for 10 minutes. The flask was then placed in an 70° C. oil bath for 5 hours with constant stirring. To the reacted mixture, styrene (3.157 g, 30.315 mmol), 4,4′-azobis(4-cyanovaleric acid) (0.194 g, 0.691 mmol), water (1.735 g) and dioxane (14.55 g) were added and again sparged with nitrogen for 10 minutes. The flask was then placed in an 70° C. oil bath for overnight with constant stirring. The final copolymer solution had solids of 23.2%.

### Part 4.2: Synthesis of Divinyl Benzene Cross-Linked Polystyrene Nanoparticles Using the Macro-RAFT Agent Prepared in Part 4.1

Macro-RAFT agent from Part 4.1 (0.504 g, 0.052 mmol) was dispersed in water (20.135 g) in a 50 mL round bottom flask. To this dispersion styrene (1.202 g, 11.545 mmol) and 2,2′-azobisisobutylronitrile (0.024 g, 0.143 mmol) were added. The flask was sealed and subsequently deoxygenated with nitrogen sparging for 5 minutes. The whole flask was immersed in an oil bath with a temperature setting of 70° C. and maintained at that temperature for 5 hours under constant magnetic stirring.

To a 50 mL round bottom flask, the obtained polystyrene latex (10.034 g), divinyl benzene (0.224 g) and 2,2′-azobisisobutylronitrile (0.032 g, 0.196 mmol) were then added, stirring to mix for 7 hours at room temperature. The flask was sealed and subsequently deoxygenated with nitrogen sparging for 5 minutes. The whole flask was immersed back in an oil bath with a temperature setting of 75° C. and maintained at that temperature for overnight under constant magnetic stirring. Transmission electron microscopy showed that the latex contained monodisperse nanoparticles, with average diameter of 110 nm, PDI of 0.078 by light scattering.

### Part 4.3: Synthesis of Polystyrene Anisotropic Nanoparticles Using the Cross-Linked Polystyrene Seeds Prepared in Part 4.2

To the cross-linked polystyrene latex from Part 4.2 (12.71 g), styrene (0.424 g, 4.075 mmol), sodium dodecyl sulphate (0.103 g, 0.358 mmol) and water (60761 g) were added, stirring to mix for 3 hours at room temperature. 4,4′-azobis(4-cyanovaleric acid) (0.069 g, 0.246 mmol) was added to the monomer swollen latex particles. The flask was sealed, stirred and subsequently deoxygenated with nitrogen sparging for 10 minutes. The whole flask was immersed in an oil bath with a temperature setting of 75° C. and maintained at that temperature for 3 hours under constant magnetic stirring. Transmission electron microscopy showed that the final latex contained monodisperse anisotropic nanoparticles with average diameter of 116 nm, PDI of 0.081 by light scattering.

### Example 5

### Synthesis of Anisotropic Nanoparticles Using Diblock Poly(AA-b-MMA/BA/AAEM) of 2-{[(butylsulfanyl)carbonothioyl]sulfanyl}propanoic acid RAFT Agent and Hexamethylene Diamine as Crosslinker

Part 5.1: Preparation of a Diblock Poly[(Methyl Methacrylate-Butyl Acrylate)ₘ-b-(acrylic acid)ₙ] Macro-RAFT Agent with Respective Degrees of Polymerization m≈12 and n≈15, in Dioxane

A solution of 2-{[butylsulfanyl)carbonothioyl]sulfanyl}propanoic acid (0.358 g, 1.50 mmol), 4,4′-azobis(4-cyanovaleric acid) (0.084 g, 0.30 mmol), acrylic acid (1.621 g, 22.5 mmol, 15 eq.) in dioxane (4.81 g) was prepared in a 100 mL round bottom flask. This was stirred magnetically and sparged with nitrogen for 10 minutes. The flask was then placed in a 70° C. oil bath for 3 hours with constant stirring. To the reacted mixture, methyl methacrylate (3.783 g, 37.78 mmol, 25 eq.), butyl acrylate (0.577 g, 4.50 mmol, 3 eq.), 4,4′-azobis(4-cyanovaleric acid) (0.085 g, 0.30 mmol) and dioxane (10.15 g) were added and again sparged with nitrogen for 10 minutes. The flask was then placed in a 70° C. oil bath and stirred overnight. Approximately 50% of the acrylic monomers reacted during this time to yield the desired length of the second block. The dioxane was removed under reduced pressure and the resulting yellow residue dissolved in methanol. The copolymer solution was finally dried under reduced pressure in a vacuum oven at 40° C. to yield a yellow powder.

Part 5.2: Synthesis of 2(acetoacetoxy)ethyl methacrylate/hexamethylene diamine Cross-Linked Acrylic Nanoparticles Using the Macro-RAFT Agent Prepared in Part 5.1

A clear solution of macro-RAFT agent from Part 5.1 (0.610 g, 0.260 mmol), sodium hydroxide (0.208 g, 5.19 mmol, 20 eq.) and water (10.4 g) was prepared in a 50 mL flask, stirring on a magnetic stirrer, which was followed by sonication in a sonic bath for 60 minutes. 4,4′-azobis(4-cyanovaleric acid) (0.202 g, 0.72 mmol) and water (4.0 g) were added, the flask was sealed and deoxygenated with nitrogen sparging for 10 minutes. A degassed mixture of methyl methacrylate (1.040 g, 10.38 mmol, 40 eq.), butyl acrylate (0.166 g, 1.30 mmol, 5 eq.) and 2(acetoacetoxy)ethyl methacrylate (0.278 g, 1.30 mmol, 5 eq.) was then added via a syringe. The reaction mixture was stirred for 3 hours and the whole flask was immersed in an oil bath with a temperature setting of 80° C. and kept at that temperature for 16 hours under constant magnetic stirring. A solution of hexamethylene diamine (0.109 g, 0.937 mmol) in water (0.991 g) was then added via a syringe pump over 18 hours at 0.06 ml/hr under constant stirring at room temperature. The final solid content of the reaction mixture was 14.6%. Transmission electron microscopy showed that the latex contained monodisperse nanoparticles with an average diameter of 46 nm.

### Part 5.3: Synthesis of Anisotropic Nanoparticles Using the Cross-Linked Acrylic Seed Particles Prepared in Part 5.2

A mixture of the seed latex from Part 5.2 (4.011 g), water (3.51 g), styrene (0.357 g, 3.428 mmol), sodium dodecyl sulphate (0.005 g) and 2,2′-azobisisobutyronitrile (0.021 g) was prepared in a 20 mL glass vial and stirred for 2 hours on a roller mixer. The vial was sealed and deoxygenated with nitrogen sparging for 5 minutes. It was then immersed in an oil bath with a temperature setting of 80° C. and maintained at that temperature for 16 hours without stirring. Transmission electron microscopy showed that the final latex contained anisotropic nanoparticles with average dimensions of 50 nm in width and 90 nm in length.

### Example 6

### Synthesis of Anisotropic Nanoparticles Using Diblock Poly(AA-b-Sty) of Dibenzyl Trithiocarbonate RAFT Agent

Part 6.1: Preparation of a Diblock Poly[(Styrene)ₘ-b-(Acrylic Acid)ₙ]₂ Macro-RAFT Agent with Respective Degrees of Polymerisation M≈15 and n≈10 in Propylene Glycol

A solution of dibenzyl trithiocarbonate (1.79 g, 6.15 mmol), 2,2′-Azobis(2-methylbutyronitrile) (0.12 g, 0.62 mmol), acrylic acid (8.87 g, 123.07 mmol) in propylene glycol (69.88 g) was prepared in a 100 mL round bottom flask. This was stirred magnetically and sparged with nitrogen for 10 minutes. The flask was then placed in a 80° C. oil bath for 3 hour with constant stirring. To the reacted mixture, 2,2′-Azobis(2-methylbutyronitrile) (0.12 g, 0.62 mmol) was added and allowed to stir for 10 minutes. Following this there was added styrene (19.23 g, 184.60 mmol), and the reaction allowed to proceed for 3 hours with constant stirring. The same amount of initiator, 2,2′-azobis(2-methylbutyronitrile) (0.12 g, 0.62 mmol) was added after 3 hours and the reaction proceeded for a further 3 hours with constant stirring. The final copolymer solution had solids of 26%.

### Part 6.2: Synthesis of Crosslinked Nanoparticles Using Diblock Poly(AA-b-Sty) of Dibenzyl Trithiocarbonate RAFT Agent

A clear solution of macro-RAFT agent from Part 6.1 (4.08 g), sodium hydroxide (0.81 g, 20.192 mmol) and water (81.52 g) were prepared in a 100 mL round bottom flask, stirring on a magnetic stirrer, followed by sonication in a ultrasonic bath for 30 minutes. To this solution styrene (9.21 g, 88.366 mmol) and sodium hydrogen carbonate (0.09 g, 1.115 mmol) were added, stirring overnight. Potassium persulphate (0.22 g, 0.797 mmol) was added to the monomer swollen micelles. The flask was sealed and subsequently deoxygenated with nitrogen sparging for 10 minutes. The whole flask was immersed in an oil bath with a temperature setting of 80° C. and maintained at that temperature for 4 hours under constant magnetic stirring. Divinyl benzene (3.73 g) and 2,2′-azobisisobutylronitrile (0.34 g, 2.099 mmol) were then added, stirring to mix for 1 hour at room temperature. The whole flask was immersed back in an oil bath with a temperature setting of 75° C. and maintained at that temperature overnight under constant magnetic stirring. The final latex particles had average diameters of 38 nm by light scattering.

### Part 6.3: Synthesis of Anisotropic Nanoparticles Using the Crosslinked Polystyrene Seed as Prepared in Part 6.2

A mixture of styrene latex from Part 6.2 (5.46 g), methyl methacrylate and butyl acrylate monomer mixture at the weight ratio of 7:3 (1.4 g) and water (13.10 g) were prepared in a 20 mL round bottom flask and stirred overnight. 2,2′-azobisisobutylronitrile (0.042 g, 0.256 mmol) was added to the monomer swollen latex particles. The flask was sealed and stirred at room temperature for 2 hours, subsequently deoxygenated with nitrogen sparging for 10 minutes. The whole flask was immersed in an oil bath with a temperature setting of 75° C. and maintained at that temperature for 4 hours under constant magnetic stirring. The resulting latex was analysed by TEM. The TEM image showed that the final latex comprised dumbbell shaped nanoparticles with a distinct protrusion derived from the expelled second stage monomers. One end of the particle is formed from polystyrene (ie. the original crosslinked seed polymer particle) while the other end is derived from the expelled monomer and is formed of polymethyl methacrylate-co-butyl acrylate.

### Example 7

### Synthesis of Anisotropic Nanoparticles Using Diblock Poly(AA-b-Sty) of 2-{[(dodecylsulfanyl)carbonothioyl]sulfanyl}propanoic acid RAFT Agent

Part 7.1: Preparation of a Diblock Poly[(Styrene)ₘ-b-(Acrylic Acid)ₙ] Macro-RAFT Agent with Respective Degrees of Polymerisation m≈30 and n≈20 in Dioxane

A solution of 2-{[(dodecylsulfanyl)carbonothioyl]sulfanyl}propanoic acid (2.13 g, 6.08 mmol), 2,2′-Azobis(2-methylbutyronitrile) (0.12 g, 0.62 mmol), acrylic acid (8.76 g, 121.57 mmol) in dioxane (69.88 g) was prepared in a 100 mL round bottom flask. This was stirred magnetically and sparged with nitrogen for 10 minutes. The flask was then placed in a 80° C. oil bath for 3 hour with constant stirring. To the reacted mixture, 2,2′-Azobis(2-methylbutyronitrile) (0.12 g, 0.62 mmol) was added and allowed to stir for 10 minutes. Following this there was added styrene (18.99 g, 182.35 mmol), and the reaction allowed to proceed for 3 hours with constant stirring. The same amount of initiator, 2,2′-Azobis(2-methylbutyronitrile) (0.12 g, 0.62 mmol) was added four consecutive times after 2 hours reaction interval. The dioxane was removed under vacuum to yield a viscous copolymer solution that had solids of 79%.

Part 7.2: Synthesis of Crosslinked Nanoparticles Using Diblock Poly(AA-b-Sty) of 2-{[dodecylsulfanyl)carbonothioyl]sulfanyl}propanoic acid RAFT Agent

A clear solution of macro-RAFT solution from Part 7.1 (5.31 g), sodium hydroxide (0.81 g, 20.192 mmol) and water (81.57 g) were prepared in a 100 mL round bottom flask, stirring on a magnetic stirrer, followed by sonication in a ultrasonic bath for 30 minutes. To this solution styrene (9.20 g, 88.292 mmol) and sodium hydrogen carbonate (0.092 g, 1.095 mmol) were added, stirring for 4 hours at room temperature. Potassium persulphate (0.23 g, 0.851 mmol) was added to the monomer swollen micelles. The flask was sealed and subsequently deoxygenated with nitrogen sparging for 10 minutes. The whole flask was immersed in an oil bath with a temperature setting of 80° C. and maintained at that temperature for 4 hours under constant magnetic stirring. Divinyl benzene (3.8 g) and 2,2′-azobisisobutylronitrile (0.34 g, 2.099 mmol) were then added, stirring to mix for 1 hour at room temperature. The whole flask was immersed back in an oil bath with a temperature setting of 75° C. and maintained at that temperature for 4 hours under constant magnetic stirring. The final latex particles had average diameters of 38 nm by light scattering.

### Part 7.3: Synthesis of Anisotropic Nanoparticles Using the Crosslinked Polystyrene Seeds as Prepared in Part 7.2

A mixture of styrene latex from Part 7.2 (5.966 g), methyl methacrylate and butyl acrylate monomer mixture at the weight ratio of 7:3 (1.5 g) and water (12.62 g) were prepared in a 20 mL round bottom flask and stirred overnight. 2,2′-azobisisobutylronitrile (0.043 g, 0.262 mmol) was added to the monomer swollen latex particles. The flask was sealed and stirred at room temperature for 2 hours, subsequently deoxygenated with nitrogen sparging for 10 minutes. The whole flask was immersed in an oil bath with a temperature setting of 75° C. and maintained at that temperature for 4 hours under constant magnetic stirring. The resulting latex was analysed by TEM. The TEM image showed that the final latex comprised dumbbell shaped nanoparticles with a distinct protrusion derived from the expelled second stage monomer. One end of the particle is formed from polystyrene (ie. the original crosslinked seed polymer particle) while the other end is derived from the expelled monomer and is formed of polymethyl methacrylate-co-butyl acrylate.

### Example 8

### Paint Evaluation of the Anisotropic Nanoparticles Synthesised in Example 6

A sample of the anisotropic nanoparticle latex of Example 6.3 (13.63 g) was blended with a sample of a typical commercially available low sheen waterborne paint, British Paints Low Sheen-Vivid White, manufactured by DuluxGroup (Australia) Pty. Ltd. (50.0 g). The blend was made homogeneous by hand shaking.

Both blended and unblended control paint samples were drawdown using a standard wet film applicator to deliver 50 μm film thickness over a glossy coated paper card. Duplicate film samples were dried at room temperature and at 80° C. for 30 minutes. Irrespective of the method of drying, both modified and control films were equivalent in appearance. No deterioration in film appearance or stability was detected.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of forming a polymer on a surface of polymer particles, the method comprising:
(i) providing a dispersion comprising a continuous aqueous phase, a dispersed organic phase comprising one or more ethylenically unsaturated monomers, and a reversible addition fragmentation chain transfer agent as a stabiliser for said organic phase;
(ii) polymerising the one or more ethylenically unsaturated monomers under the control of the reversible addition fragmentation chain transfer agent to form an aqueous dispersion of seed polymer particles;
(iii) crosslinking the seed polymer particles;
(iv) swelling the crosslinked seed particles with one or more ethylenically unsaturated monomers to form an aqueous dispersion of monomer swollen crosslinked seed polymer particles;
(v) increasing a temperature of the monomer swollen crosslinked seed polymer particles to expel at least some of the monomer therein onto a surface of the particles; and
(vi) polymerising at least the expelled monomer to form the polymer on the surface of the particles.

2. The method according to claim 1, wherein increasing the temperature of the monomer swollen crosslinked seed polymer particles expels at least some of the monomer therein onto substantially the entire surface of the particles, and polymerisation of at least the expelled monomer results in the formation of core-shell polymer particles.

3. The method according to claim 1, wherein increasing the temperature of the monomer swollen crosslinked seed polymer particles expels at least some of the monomer therein only onto a proportion of the surface of the particles, and polymerisation of at least the expelled monomer results in the formation of non-core-shell polymer particles.

4. The method according to claim 3, wherein the monomer is expelled on to no more than about 70% of the particle surface.

5. The method according to claim 1, wherein the seed polymer particles have a largest dimension of no more than about 50 nm.

6. The method according to claim 1, wherein the so formed polymer particles have a largest dimension of no more than about 100 nm.

7. The method according to claim 1, wherein the ethylenically unsaturated monomers are selected from those of formula (I): 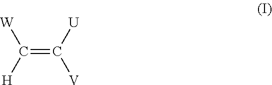
where U and W are independently selected from —CO₂H, —CO₂R¹, —COR¹, —CSR¹, —CSOR¹, —COSR¹, —CONH₂, —CONHR¹, —CONR¹₂, hydrogen, halogen and optionally substituted C₁-C₄ alkyl, or U and W form together a lactone, anhydride or imide ring that may itself be optionally substituted, wherein the substituents are independently selected from the group consisting of hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹; and
V is selected from hydrogen, R¹, —CO₂H, —CO₂R¹, —COR¹, —CSR¹, —CSOR¹, —COSR¹, —CONH₂, —CONHR¹, —CONR¹₂, —OR¹, —SR¹, —O₂CR¹, —SCOR¹, and —OCSR¹;
where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

8. The method according to claim 1, wherein the reversible addition fragmentation chain transfer agent is of general formula (II): 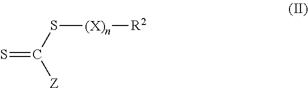
where each X is independently a polymerised residue of an ethylenically unsaturated monomer, n is an integer ranging from 0 to 100, and R² and Z are each groups independently selected such that the agent functions as a reversible addition fragmentation chain transfer agent in the polymerisation of the one or more ethylenically unsaturated monomers.

9. The method according to claim 8, wherein R² is selected from alkyl, alkylaryl, arylalkyl, alkoxyaryl and alkoxyheteroaryl, each of which is optionally substituted with one or more hydrophilic groups.

10. The method according to claim 8, wherein Z is selected from optionally substituted alkoxy, optionally substituted aryloxy, optionally substituted alkyl, optionally substituted aryl, optionally substituted heterocyclyl, optionally substituted arylalkyl, optionally substituted alkylaryl, optionally substituted alkylthio, optionally substituted arylalkylthio, dialkoxy- or diaryloxy-phosphinyl [—P(═O)OR⁴₂], dialkyl- or diaryl-phosphinyl [—P(═O)R⁴₂], optionally substituted acylamino, optionally substituted acylimino, optionally substituted amino, R²—(X)ₙ—S—, and a polymer chain formed by any mechanism; wherein R⁴ is selected from optionally substituted C₁-C₁₈ alkyl, optionally substituted C₂-C₁₈ alkenyl, optionally substituted aryl, optionally substituted heterocyclyl, optionally substituted aralkyl, and optionally substituted alkaryl.

11. The method according to claim 1, wherein the crosslinking the seed polymer particles is promoted using one or more multi-ethylenically unsaturated monomers selected from ethylene glycol di(meth)acrylate, triethylene glycol di(meth)acrylate, tetraethylene glycol di(meth)acrylate, 1,3-butylene glycol di(meth)acrylate, trimethylolpropane tri(meth)acrylate, 1,4-butanediol di(meth)acrylate, neopentyl glycol di(meth)acrylate, 1,6-hexanediol di(meth)acrylate, pentaerythritol di(meth)acrylate, pentaerythritol tri(meth)acrylate, pentaerythritol tetra(meth)acrylate, glycerol di(meth)acrylate, glycerol allyloxy di(meth)acrylate, 1,1,1-tris(hydroxymethyl)ethane di(meth)acrylate, 1,1,1-tris(hydroxymethyl)ethane tri(meth)acrylate, 1,1,1-tris(hydroxymethyl)propane di(meth)acrylate, 1,1,1-tris(hydroxymethyl)propane tri(meth)acrylate, triallyl cyanurate, triallyl isocyanurate, triallyl trimellitate, diallyl phthalate, diallyl terephthalte, divinyl benzene, methylol (meth)acrylamide, triallylamine, oleyl maleate, glyceryl propoxy triacrylate, allyl methacrylate, methacrylic anhydride and methylenebis(meth) acrylamide.

12. The method according to claim 1, wherein the crosslinking the seed polymer particles is promoted using one or more ethylenically unsaturated monomers selected from acetoacetoxyethyl methacrylate, glycidyl methacrylate, N-methylolacrylamide, (isobutoxymethyl)acrylamide, hydroxyethyl acrylate, t-butyl-carbodiimidoethyl methacrylate, acrylic acid, γ-methacryloxypropyltriisopropoxysilane, 2-isocyanoethyl methacrylate and diacetone acrylamide.

13. The method according to claim 1, wherein the swelling in step (iv) comprises saturating the crosslinked seed particles with one or more ethylenically unsaturated monomers.

14. The method according to claim 1, wherein increasing the temperature in step (v) comprises increasing the temperature of the swollen crosslinked seed particles by at least 40° C.

15. A coating, adhesive, filler, primer, sealant, pharmaceutical, cosmetic, diagnostic, or therapeutic product comprising the polymer particles prepared in accordance with claim 1.

16. Polymer particles capable of being dispersed in a liquid, the particles comprising two polymer regions of different molecular composition, wherein one of the polymer regions is a crosslinked reversible addition fragmentation chain transfer polymer having covalently bound to its surface reversible addition fragmentation chain transfer polymer chains that stabilise the polymer particles when they are dispersed in the liquid.

17. The polymer particles according to claim 16 which are self stabilising.

18. The polymer particles according to claim 16, wherein the liquid is an aqueous liquid.

19. The method according to claim 11, wherein the crosslinking the seed polymer particles is promoted using additionally one or more ethylenically unsaturated monomers selected from acetoacetoxyethyl methacrylate, glycidyl methacrylate, N-methylolacrylamide, (isobutoxymethyl)acrylamide, hydroxyethyl acrylate, t-butyl-carbodiimidoethyl methacrylate, acrylic acid, γ-methacryloxypropyltriisopropoxysilane, 2-isocyanoethyl methacrylate and diacetone acrylamide.

20. The method according to claim 10, wherein R² is selected from alkyl, alkylaryl, arylalkyl, alkoxyaryl and alkoxyheteroaryl, each of which is optionally substituted with one or more hydrophilic groups.
